(19) United States
(12) Patent Application Publication
(10) Pub. No.: US 2023/0340625 A1
(43) Pub. Date: Oct. 26, 2023

(54) METHOD AND SYSTEM FOR DETECTING AND TREATING EXPOSURE TO AN INFECTIOUS PATHOGEN

(71) Applicant: Sun Genomics, Inc., San Diego, CA (US)

(72) Inventor: Suneer Jain, San Diego, CA (US)

(21) Appl. No.: 17/918,535

(22) PCT Filed: Apr. 13, 2021
(86) PCT No.: PCT/US2021/027139

§ 371 Date: Oct. 12, 2022

Related U.S. Application Data
(60) Provisional application No. 63/032,416, filed on May 29, 2020.
(60) Provisional application No. 63/009,402, filed on Apr. 13, 2020.

(51) Int. Cl. C12Q 1/70; G01N 33/569; A61K 35/745; A61K 35/747; A61K 35/742; A61K 36/064; A61K 35/744; A61K 31/375; A61K 31/7048; A61K 36/752; A61K 31/352; A61K 36/82; A61K 45/06; A61P 31/14

(52) U.S. Cl. CPC C12Q 1/701; G01N 33/56983; A61K 35/745; A61K 35/747; A61K 35/742; A61K 36/064; A61K 35/744; A61K 31/375; A61K 31/7048; A61K 36/752; A61K 31/352; A61K 36/82; A61K 45/06; A61P 31/14; G01N 2333/165

(57) ABSTRACT

Disclosed herein is a method and system for detecting exposure of a patient to an infectious pathogen, as well as customized treatment of an infected patient by analysis and classification of the patient's microbiome. The methodology described herein provides detection, analysis, and treatment of a subject exposed to an infectious pathogen.

### CROSS-REFERENCE TO RELATED APPLICATIONS

This application claims benefit of priority under 35 U.S.C. § 119(e) of U.S. Provisional Patent Application Ser. No. 63/032,416, filed May 29, 2020; and U.S. Provisional Patent Application Ser. No. 63/009,402, filed Apr. 13, 2020. The disclosure of the prior applications are considered part of and are incorporated by reference in the disclosure of this application.

### INCORPORATION OF SEQUENCE LISTING

The material in the accompanying sequence listing is hereby incorporated by reference into this application. The accompanying sequence listing text file, name SUN1170-2WO_SL.TXT, was created on Apr. 13, 2021, and is 11 kb. The file can be accessed using Microsoft Word on a computer that uses Windows OS.

### BACKGROUND OF THE INVENTION

### Field of Invention

The present invention relates generally to infectious pathogens and more particularly to a method and system for detecting and treating a subject exposed to an infectious pathogen and/or having a pathogenic infection.

### Background Information

About 100 trillion microorganisms live in and on the human body vastly outnumbering the body's approximately 10 trillion human cells. These normally harmless viruses, bacteria and fungi are referred to as commensal or mutualistic organisms. Commensal and mutualistic organisms help keep our bodies healthy in many ways. Together all of the microorganisms living in and on the body—commensal, mutualistic and pathogenic—are referred to as the microbiome and their equilibrium and associated metabolome is closely linked to an individual's health status and vice-versa.

Advances in nucleic acid sequencing has created an opportunity to quickly and accurately identify and profile the microbiome inhabiting the gut and subcutaneous tissue. The optimal flora also interacts with the host immune system in a synergistic way further propagating its health benefits. The associated metabolome of individuals can also be profiled either by a mass-spectrometry based system or using genomics-based metabolome modeling and flux-balance analysis and used to make a healthy metabolome profile. All these methodologies can be used to dissect the complexity of microbial communities.

Detection of SARS-CoV-2, the causative agent of COVID-19, at an early stage of the disease is important at this unprecedented time of the Pandemic. Recent studies have demonstrated the presence of SARS-CoV-2 in stool samples and the accuracy of tests in detecting nucleic acids in stool samples. Several clinical cases reported positive results up to 12 days of duration time, regardless of age and gender. RT-PCR tests showed shedding of the virus in stool was evident for at least five weeks after the respiratory samples turned negative.

SARS-CoV-2 uses angiotensin converting enzyme (ACE2) as a viral receptor to enter the host. ACE2 shows high levels of expression in the gastrointestinal system compared to other systems. In some cases, primary symptoms were gastrointestinal symptoms like diarrhea, nausea and vomiting and abdominal pain was reported more frequently in patients admitted to the intensive care unit. SARS-CoV-2 can also be detected in fecal specimens of asymptomatic patients. Shedding of SARS-CoV-2 in stool points to a potential fecal-oral route of transmission for COVID-19.

Detection of infectious pathogens, such as SARS-CoV-2, along with analysis of the microbiome of an infected patient, allows for customized treatment options, such as administration of a probiotic, pre-biotic and/or a metabolite of the gut microbiome, to assist in disease prevention and/or speeding disease recovery.

### SUMMARY OF THE INVENTION

The present invention is directed to a method and system for detecting exposure of a patient to an infectious pathogen, as well as customized treatment of an infected patient by analysis and classification of the patient's microbiome.

Accordingly, in one embodiment, the invention provides a method of detecting an infectious pathogen in a subject and optionally treating the subject. The method includes detecting exposure to a pathogen in a subject, analyzing the microbiome of the subject and identifying opportunistic pathogens in the subject that indicate a dysbiosis or potential onset/recovery of disease symptoms, and optionally treating the subject with a therapeutic composition. In some aspects, the therapeutic composition includes a probiotic, pre-biotic and/or metabolite of the gut microbiome. In some aspect, the therapeutic composition is customized to the patient based on the analysis of the patient's microbiome.

In another embodiment, the invention provides a therapeutic formulation, e.g., therapeutic composition, for treatment of a subject exposed to or diagnosed with an infection disease. The formulation includes a naturally occurring product or derivative thereof; and optionally a customized probiotic, pre-biotic and/or metabolite of the gut microbiome. In some aspects, the therapeutic formulation includes a synthetically derived natural product or an isolated and purified naturally occurring product in combination with a customized probiotic, pre-biotic and/or metabolite of the gut microbiome, such as a probiotic including one or more microorganisms. In various aspects, the therapeutic formulation treats an infectious disease or otherwise inhibits and/or ameliorates symptoms associated with the infectious disease to promote recovery. In some aspects, the therapeutic formulation treats dysbiosis of a subject exposed to or diagnosed with an infectious disease to inhibit and/or ameliorate symptoms associated with the infectious disease to promote recovery. In some aspects, the therapeutic composition includes, or is used in combination with a drug, such as an antiviral agent, that is conventionally used to treat a viral and/or pathogenic infection.

In yet another embodiment, the invention provides a method of treating a subject exposed to or diagnosed with an infectious disease. The method includes administering the subject a therapeutic composition of the invention.

In still another embodiment, the invention provides a method for screening a subject for exposure to an infectious pathogen and treating the subject where the subject has been exposed to the infectious pathogen and/or exhibits symptoms associated with pathogenic infection.

In some aspects, the method includes screening a screening a subject for a previous exposure to a virus using an antibody assay, and where the antibody assay is negative, screening the subject for the virus using a PCR based assay and administering the subject a therapeutic composition of the invention.

In some aspects, the method includes screening a subject for a previous exposure to a virus using an IgG/IgM specific antibody assay, wherein if the subject is IgM negative, the subject is screened for the virus via a PCR based assay and administered the therapeutic composition of the invention where the PCR based assay is positive and then rescreened using the IgG/IgM specific antibody assay after about 3 to 21 days, and wherein if the subject is IgM positive, the subject is administered the therapeutic composition of therapeutic composition of the invention and then rescreened using the IgG/IgM specific antibody assay after about 3 to 21 days.

In some aspects, the method includes screening a subject for a viral infection using a PCR based assay, wherein if the PCR based assay is positive the subject is administered the therapeutic composition of any one of claims 22 to 35 and then rescreened using the PCR based assay after about 3 to 21 days, and wherein if the PCR based assay is negative, the subject is screened for a previous exposure to the virus using an IgG/IgM specific antibody assay, and wherein if the subject is IgM negative, the subject is screened for risk of infecting another subject via a PCR based test and administered the therapeutic composition of the invention where the PCR based assay is positive and then rescreened using the IgG/IgM specific antibody assay after about 3 to 12 days, and wherein if the subject is IgM positive, the subject is administered the therapeutic composition of the invention and then rescreened using the IgG/IgM specific antibody assay after about 3 to 21 days.

In another embodiment, the invention provides a method for detecting SARS-CoV-2 in a biological sample, such as a stool sample. In one aspect, the method is a PCR based assay as described in Example 1.

In some aspects, the method includes:
obtaining a biological sample comprising ribonucleic acids;
reverse transcribing the ribonucleic acids to obtain cDNA;
contacting the cDNA with a first and/or second primer set, and a DNA polymerase to produce a first and/or second PCR product, wherein the first primer set comprises SEQ ID NOs: 1 and 2 and the second primer set comprises SEQ ID NOs: 5 and 6;
hybridizing to the first PCR product a first nucleic acid probe comprising SEQ ID NO: 3 and/or SEQ ID NO: 4, and/or hybridizing to the second PCR product a second nucleic acid probe comprising SEQ IN NO: 7 and/or 8; and
detecting hybridization of the first nucleic acid probe to the first PCR product and/or detecting hybridization of the second nucleic acid probe to the second PCR product,
wherein hybridization of the first nucleic acid probe to the first PCR product, hybridization of the second nucleic acid probe to the second PCR product, is indicative of the presence of SARS-CoV-2 nucleic acids in the biological sample.

In some aspects, method includes:
contacting the cDNA with a control primer set, and a DNA polymerase to produce a control PCR product, wherein the control primer set comprises SEQ ID NOs: 9 and 10;
hybridizing to the control PCR product a control nucleic acid probe comprising SEQ ID NO: 11 and/or SEQ ID NO: 12; and
detecting hybridization of the control nucleic acid probe to the control PCR product.

In another embodiment, the invention provides a kit for detecting SARS-CoV-2. The kit includes first and/or second primer set, wherein the first primer set comprises SEQ ID NOs: 1 and 2 and the second primer set comprises SEQ ID NOs: 5 and 6, a first nucleic acid probe comprising SEQ ID NO: 3 and/or SEQ ID NO: 4, and/or a second nucleic acid probe comprising SEQ IN NO: 7 and/or 8; and optionally reagents for conducting a reverse transcription-polymerase chain reaction using a) and b). In some aspects, the kit further includes a control primer set, wherein the control primer set comprises SEQ ID NOs: 9 and 10, and a control nucleic acid probe comprising SEQ ID NO: 11 and/or SEQ ID NO: 12.

### BRIEF DESCRIPTION OF THE DRAWINGS

FIG. 1 is an image of a screenshot listing opportunistic pathogens associated with pathogenic infection, such as infection by SARS-CoV-2, of a subject in aspects of the invention.

FIG. 2 is an image of a screenshot listing opportunistic pathogens associated with pathogenic infection, such as infection by SARS-CoV-2, of a subject in aspects of the invention.

FIG. 3 is an image of a screenshot listing opportunistic pathogens associated with pathogenic infection, such as infection by SARS-CoV-2, of a subject in aspects of the invention.

FIG. 4 is an image of a screenshot listing opportunistic pathogens associated with pathogenic infection, such as infection by SARS-CoV-2, of a subject in aspects of the invention. Of particular note is Serratia marcescens, an opportunist pathogen (harmful microbe) that can be associated with hospital-acquired infections.

### DETAILED DESCRIPTION OF THE INVENTION

The present invention provides a method and system for detecting exposure of a patient to an infectious pathogen, as well as customized treatment of an infected patient by analysis and classification of the patient's microbiome. The invention utilizes a method for detecting infectious pathogens, such as SARS-CoV-2, in a biological sample via a PCR based assay, as well as microbiome analysis to produce customized therapeutic compositions for prevention and/or treatment of pathogenic infection.

In some aspects, microbiome analysis utilizes a universal method for extracting nucleic acid molecules from a diverse population of one or more types of microbes in a sample. In various aspects, the types of microbes include, but are not limited to, gram-positive bacteria, gram-positive bacterial spores, gram-negative bacteria, archaea, protozoa, helminths, algae, fungi, fungal spores, viruses, viroids, bacteriophages, and rotifers. In some aspects, the diverse population is a plurality of different microbes of the same type, e.g., gram-positive bacteria. In some aspects, the diverse population is a plurality of different types of microbes, e.g., bacteria (gram-positive bacteria, gram-positive bacterial spores and/or gram-negative), fungi, viruses, and bacteriophages.

Before the present compositions and methods are described, it is to be understood that this invention is not limited to the particular methods and systems described, as such methods and systems may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Accordingly, in one embodiment, the invention provides a method of detecting an infectious pathogen in a subject and optionally treating the subject. The method includes detecting exposure to a pathogen in a subject, analyzing the microbiome of the subject and identifying opportunistic pathogens in the subject that indicate a dysbiosis or potential onset/recovery of disease symptoms, and optionally treating the subject with a therapeutic composition. In some aspects, the therapeutic composition includes a probiotic, pre-biotic and/or metabolite of the gut microbiome. In some aspect, the therapeutic composition is customized to the patient based on the analysis of the patient's microbiome.

As used herein, the term “microbiome” refers to microorganisms, including, but not limited to bacteria, phages, viruses, and fungi, archaea, protozoa, amoeba, or helminths that inhabit the gut of a subject.

As used herein, the terms “microbial”, “microbe”, and “microorganism” refer to any microscopic organism including prokaryotes or eukaryotes, spores, bacterium, archeaebacterium, fungus, virus, or protist, unicellular or multicellular.

As used herein, the term “subject” or “patient” includes humans and non-human animals. The term “non-human animal” includes all vertebrates, e.g., mammals and non-mammals, such as non-human primates, horses, sheep, dogs, cows, pigs, chickens, and other veterinary subjects and test animals.

It will be appreciated that detection of an infectious pathogen may be performed by any number of detection modalities known in the art. In some aspects, detection of a pathogen includes use of a PCR based assay to detect a nucleic acid. In various aspects, DNA and/or RNA can be separated and analyzed by molecular methods, such as whole or targeted transcriptomics, reverse transcriptase qPCR (RT-qPCR), qPCR, expression microarrays or other techniques known to the art. In one aspect, detection is of SAR-CoV-2 using an RT-qPCR method as set forth in Example 1.

As used herein, the terms “polynucleotide”, “nucleic acid” and “oligonucleotide” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cfDNA and cell-free RNA (cfRNA), nucleic acid probes, and primers. A polynucleotide may include one or more modified nucleotides, such as methylated nucleotides and nucleotide analogs.

In various aspects, analysis can be of any nucleic acid. This nucleic acid can be of any length, as short as oligos of about 5 bp to as long a megabase or even longer. A “nucleic acid molecule” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule.

A single-stranded nucleic acid molecule is “complementary” to another single-stranded nucleic acid molecule when it can base-pair (hybridize) with all or a portion of the other nucleic acid molecule to form a double helix (double-stranded nucleic acid molecule), based on the ability of guanine (G) to base pair with cytosine (C) and adenine (A) to base pair with thymine (T) or uridine (U). For example, the nucleotide sequence 5′-TATAC-3′ is complementary to the nucleotide sequence 5′-GTATA-3′.

As used herein “hybridization” refers to the process by which a nucleic acid strand joins with a complementary strand through base pairing. Hybridization reactions can be sensitive and selective so that a particular sequence of interest can be identified even in samples in which it is present at low concentrations. In an in vitro situation, suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature. For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 mg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

As used herein, the terms “pathogen” and “infectious pathogen” are used interchangeably. In various aspects, a pathogen may be a bacterial, fungal, parasitic or viral pathogen. In some aspects, the pathogen is a viral pathogen, such as coronavirus, Zika virus, influenza virus or Ebola virus. In some aspects, the coronavirus is Coronavirus Disease 2019 (COVID-19), SARS associated coronavirus (SARS-CoV), or Middle East respiratory syndrome coronavirus (MERS-CoV). In some aspects, the coronavirus is SARS-CoV-2.

As discussed above, in addition to the infectious pathogens already mentioned herein, it is understood that the system and method of the invention can be used to detect any number pathogens including, but not limited to Bacillus anthracis (anthrax), Yersinia pestis (pneumonic plague), Franciscella tularensis (tularemia), Brucella suis, Brucella abortus, Brucella melitensis (undulant fever), Burkholderia mallei (glanders), Burkholderia pseudomalleii (melioidosis), Salmonella typhi (typhoid fever), Rickettsia typhii (epidemic typhus), Rickettsia prowasekii (endemic typhus) and Coxiella burnetii (Q fever), Rhodobacter capsulatus, Chlamydia pneumoniae, Escherichia coli, Shigella dysenteriae, Shigella flexneri, Bacillus cereus, Clostridium botulinum, Coxiella burnetti, Pseudomonas aeruginosa, Legionella pneumophila, and Vibrio cholerae.

In some aspects, the pathogen is a biological warfare fungus, such as Coccidioides immitis (Coccidioidomycosis).

Additional examples of (−)-strand RNA viruses that may be detected include arenaviruses (e.g., sabia virus, lassa fever, Machupo, Argentine hemorrhagic fever, flexal virus), bunyaviruses (e.g., hantavirus, nairovirus, phlebovirus, hantaan virus, Congo-crimean hemorrhagic fever, rift valley fever), and mononegavirales (e.g., filovirus, paramyxovirus, ebola virus, Marburg, equine morbillivirus).

Additional examples of (+)-strand RNA viruses that may be detected include picornaviruses (e.g., coxsackievirus, echovirus, human coxsackievirus A, human echovirus, human enterovirus, human poliovirus, hepatitis A virus, human parechovirus, human rhinovirus), astroviruses (e.g., human astrovirus), calciviruses (e.g., chiba virus, chitta virus, human calcivirus, norwalk virus), nidovirales (e.g., human coronavirus, human torovirus), flaviviruses (e.g., dengue virus 1-4, Japanese encephalitis virus, Kyanasur forest disease virus, Murray Valley encephalitis virus, Rocio virus, St. Louis encephalitis virus, West Nile virus, yellow fever virus, hepatitis C virus) and togaviruses (e.g., Chikugunya virus, Eastern equine encephalitis virus, Mayaro virus, O'nyong-nyong virus, Ross River virus, Venezuelan equine encephalitis virus, Rubella virus, hepatitis E virus).

Because different types of microbes have different compositions and mechanisms to protect their own genetic material it is often difficult to extract the genetic material from one type of microbe without compromising the ability to also extract the genetic material of another type of microbe in the same biological sample. The present invention, however, utilizes techniques that allow the extraction of genetic material from different types of microbes in a sample without sacrificing the amount of genetic material that can be obtained from one type of microbe by extracting the genetic material of another type of microbe in the same sample. As will be appreciated, this is particularly advantageous for extraction of nucleic acid from a diverse population of microbes in performing genomic analysis of a microbiome of a patient.

In various aspects, the methodology of the present invention includes extracting and analyzing nucleic acids present in a biological sample obtained from a subject to detect a pathogen. The methodology also includes extracting and analyzing nucleic acids present in a biological sample obtained from a subject to perform microbiome analysis.

In various aspects, the sample obtained from the subject that includes microbes is a biological sample. Similarly, the sample obtained from the subject used to detect a pathogen is also a biological sample. Examples of biological samples include tissue samples, blood samples, plasma samples, cerebrospinal fluid samples, urine samples, gut and/or fecal samples, samples of material obtained from the digestive tract, biological secretions (e.g., semen, vaginal secretions, breast milk, tears, saliva) and the like. Solid samples may be liquefied or mixed with a solution, and then genetic material of the microbes present in the liquefied sample, mixture, or solution obtained from the mixture may be extracted in accordance with the present invention. The extracted genetic material may be subjected to further processing and analysis such as purification, amplification, and sequencing.

In some aspects, a sample is a gut or fecal sample obtained by non-invasive or invasive techniques such as biopsy of a subject. In one aspect, the term “sample” refers to any preparation derived from fecal matter or gut tissue of a subject. For example, a sample of material obtained using the non-invasive method described herein can be used to isolate nucleic acid molecules or proteins for the methods of the present invention.

In some embodiments, the extracted genetic material is subjected to metagenomics analysis to, for example, identify the one or more types of microbes in the sample from which the genetic material was extracted for microbiome analysis. In additional embodiments, full whole genome shotgun sequencing can be performed on prepared extracted nucleic acid material from human fecal samples. Preparations include nucleic acid clean up reactions to remove organic solvents, impurities, salts, phenols, and other process inhibiting contaminants. Additional preparations include nucleic acid library prep from each sample where the gDNA is subject to modifications and/or amplifications to prep the sample for sequencing on a sequencing platform such as massively parallel sequencing by synthesis, nanopore, long read, and/or CMOS electronic, sequencing methods. In some aspects, nucleic acid is extracted and processed for microbiome analysis as described in International Patent Application No. PCT/US2019/058224, the content of which is incorporated by reference in its entirety.

In the various aspects discussed herein, processing steps may include, RNA or DNA clean-up, fragmentation, separation, or digestion; library or nucleic acid preparation for downstream applications, such as PCR, qPCR, digital PCR, or sequencing; preprocessing for bioinformatic QC, filtering, alignment, or data segregation; metagenomics or human genomic bioinformatics pipeline for microbial species taxonomic assignment; and other organism alignment, identification, and variant interpretation.

In certain aspects, the method of the present invention uses stool samples obtained from a subject for DNA extraction and microbiome analysis. In some aspects, the extracted genetic material is subjected to further processing and analysis, such as purification, amplification and sequencing. In various aspects, the method furth includes subjecting the extracted genetic material to metagenomics analysis to, for example, to identify the one or more types of organisms in the sample from which the genetic material was extracted.

In some aspects the database that the metagenomic analysis will utilize has been customized for a specific purpose of identifying and taxonomically assigning, within the appropriate phylogeny, the nucleic acids with relative abundances of organisms or components of organisms ingested by humans or other animals. In some aspects, an additional data table or database may be used as a lookup of the relative abundances of organisms to determine macronutrient content of an organism's gut sample as a representation of their diet. In some embodiments this macronutrient breakdown may include fats, carbohydrates, proteins, vitamins minerals, and subcomponents of any macronutrients.

In some aspects, extracted and purified genetic material is prepared for sequencing using Illumina index adaptors and checked for sizing and quantity. A range from 1000 or greater reads of sequencing for short insert methods can be used for this method. Large insert methods such as Pac Bio™, Nanopore™, or other next generation sequencing methods can use <1000 sequencing reads. Bioinformatics quality filtering was performed before taxonomy assignment. Quality trimming of raw sequencing files may include removal of sequencing adaptors or indexes; trimming 3′ or 5′ end of reads based on quality scores (Q20>), basepairs of end, or signal intensity; removal of reads based on quality scores, GC content, or non-aligned basepairs; removal of overlapping reads at set number of base pairs. Alignment of processed sequencing files was done using a custom microbial genome database consisting of sequences from Refseq™, Greengeens™, HMP™, NCBI™, PATRIC™, or other public/private data repositories or in-house data sets. This database may be used as full genome alignment scaffold, k-mer fragment alignment, or other schemes practiced in the art of metagenomics and bioinformatics. Based off the number of sequencing reads/fragments that match the database genomes we assign a taxonomic identity that is common or unique to the organism. This identifier can be a barcode, nucleotide sequence, or some other computational tag that will associate the matching sequencing read to an organism or strain within a taxonomic group. Some identifiers will be of higher order and would identify domain, kingdom, phylum, class, order, family, or genus of the organism.

In various aspects, the present invention is able to identify the organism at the lowest order of strain within a species.

In some aspects, sequencing of the nucleic acid from the sample is performed using whole genome sequencing (WGS) or rapid WGS (rWGS). In some aspects, targeted sequencing is performed and may be either DNA or RNA sequencing. The targeted sequencing may be to a subset of the whole genome. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain aspects, clonally amplified DNA templates or single DNA molecules are sequenced in a massively parallel fashion within a flow cell (e.g., as described in WO 2014/015084). In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (e.g, singleplex sequencing) or DNA from multiple samples can be pooled and sequenced as indexed genomic molecules (e.g, multiplex sequencing) on a single sequencing run, to generate up to several hundred million reads of DNA sequences. Commercially available platforms include, e.g, platforms for sequencing-by-synthesis, ion semiconductor sequencing, pyrosequencing, reversible dye terminator sequencing, sequencing by ligation, single-molecule sequencing, sequencing by hybridization, and nanopore sequencing. In some aspects, the methodology of the disclosure utilizes systems such as those provided by Illumina, Inc, (HiSeq™ X10, HiSeq™ 1000, HiSeq™ 2000, HiSeq™ 2500, HiSeq™ 4000, NovaSeq™ 6000, Genome Analyzers™, MiSeq™ systems), Applied Biosystems Life Technologies (ABI PRISM™ Sequence detection systems, SOLiD™ System, Ion PGM™ Sequencer, ion Proton™ Sequencer).

In some aspects, the invention includes identification and/or analysis of one or more microbes contained within a biological sample of a sample obtained from a subject that has been exposed to a pathogen. In some aspects, the invention includes identification and/or analysis of one or more microbes contained within a biological sample of a sample obtained from a subject that is, or has been infected with a pathogen. In some aspects, the invention includes identification and/or analysis of one or more microbes contained within a biological sample of a sample obtained from a subject that is, or has been infected with a pathogen as determined by a RT-qPCR assay as described in Example 1.

In some aspects, the invention includes detection of viruses, phages, or other microbes that are RNA based, such as, but not limited to, influenza, MERS, SARS, and SARS-CoV-2 (an RNA virus).

In some aspects, the detection is of a virus, such as SARS-CoV-2 via a detection method utilizing PCR, such as RT-qPCR and one or more of: differentiation from viruses of the Orthomyxoviridae family; and/or differentiation from other microbes that can infect the upper or lower respiratory tract that have symptoms similar to that of SARS-CoV-2 that may be from other virus families or other microbe kingdom or phyla, such as influenza, bacterial Pseudomonas fragi, Pseudomonas aureginosa, Klebsiella species, Morganella or other opportunistic pathogens of the airway or gut; and/or detection and differentiation between mutations and strains of the virus (e.g., SARS-CoV-2).

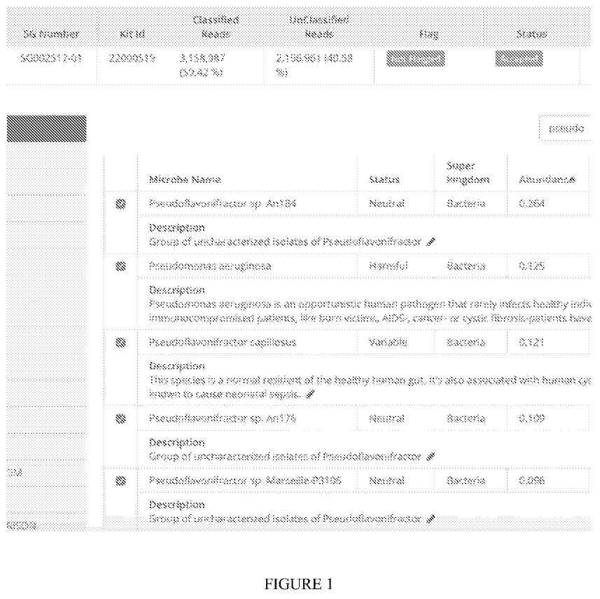

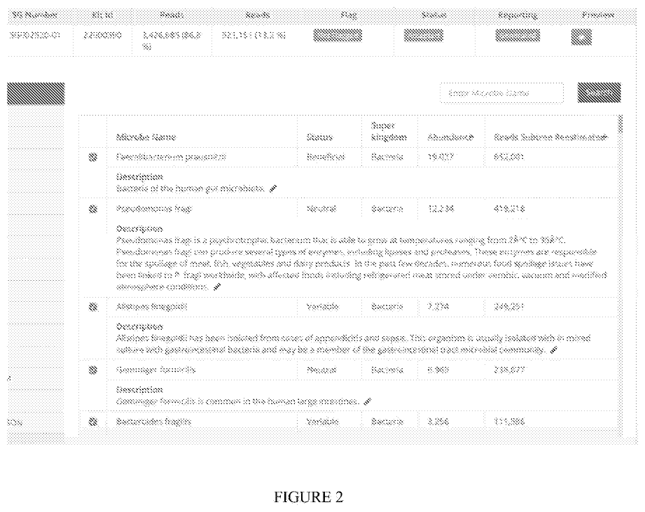

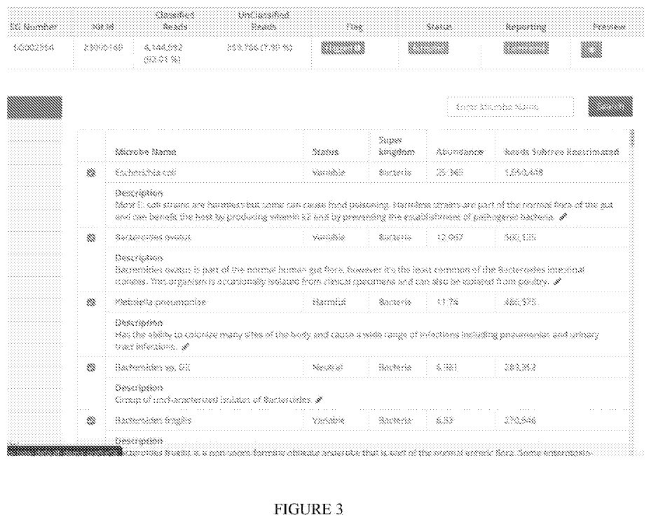

In various aspects, opportunistic microbes include any combination of those shown in FIGS. 1-4 or Tables 6-9.

In various aspects, this information could be used to guide therapeutic or natural probiotic/herbal prebiotic remedy to pathogenic exposure or infection. Based on the result from the analysis, one could use software like bioinformatics and metagenomics to understand where to target such remedy.

As such, the invention further provides a therapeutic formulation for treatment of a subject exposed to or diagnosed with an infection disease. The formulation includes a naturally occurring product or derivative thereof; and optionally a customized probiotic, pre-biotic and/or metabolite of the gut microbiome. In some aspects, the therapeutic formulation includes a synthetically derived natural product or an isolated and purified naturally occurring product in combination with a customized probiotic, pre-biotic and/or metabolite of the gut microbiome, such as a probiotic including one or more microorganisms. In various aspects, the therapeutic formulation treats an infectious disease or otherwise inhibits and/or ameliorates symptoms associated with the infectious disease to promote recovery. In some aspects, the therapeutic formulation treats dysbiosis of a subject exposed to or diagnosed with an infectious disease to inhibit and/or ameliorate symptoms associated with the infectious disease to promote recovery. In some aspects, the therapeutic composition includes, or is used in combination with a drug, such as an antiviral agent, that is conventionally used to treat a viral and/or pathogenic infection.

In another embodiment, the invention provides a method of treating a subject exposed to or diagnosed with an infectious disease. The method includes administering the subject a therapeutic composition of the invention.

In the case of SARS-CoV-2, a customized therapeutic formulation may target one or more viral components or pathways to prevent or ameliorate infection or infection related symptoms. For example, ingredients of the formulation may target of the following for remedy: virus spike surface proteins; cell or virus membrane proteins and receptors such as ACE2 and endocytosis; intra or extracellular signaling pathways such as ACE2, MAP2K; proteolysis such as 3C-like protease inhibition; translation of RNA from virus and RNA replication; and/or packaging of virus and release from cells.

In current therapeutic solutions, multiple entry and infection modes may be targeted at the same time. While some medical care may provide an antiviral drug (e.g., Remdesivir) to block RNA transcription machinery and an antibiotic (e.g., Amoxicillin) to deplete any bacterial opportunistic pathogens, the current invention is to provide natural or naturally derived products and extracts, e.g., beneficial microbes, metabolites, plant extracts, vitamins, minerals, enzymes, co-enzymes and the like. The formulation of the invention can be used in conjunction with the diagnostic/testing or optionally used independently as a preventative or natural measure to inhibit viral infection exacerbation.

In various aspects, the following formula items may be used individually or in any combination with one another to represent the formula.

Hesperidin to inhibit viral replication and entry into the cell via RDS spike protein mediated PD-ACE2 (optionally replaceable by other derivatives of Citrus, such as Vitamin C or ascorbic acid)

Quercetin and its analogs, such as quercetin 3-β-O-d-glucoside where quercetin can be naturally extracted or derived, for example, from juniper berries, onions, blueberries or other food items that contain flavonoids. In one aspect, its effect may be to inhibit the viral update of bound viral epitopes to the cell surface to inhibit fusion and deposit of viral machinery into the host cell. In one aspect, its effect may be to inhibit proteolysis that would otherwise enable proper scaffolding and packing of the virus should it have successfully infected the host cell such that replication of the invading virus is inhibited. Use of quercetin may be optionally replaced or augmented with other flavonoids.

Compounds that stimulate the immune system to help repair or prevent injury/inflammation overload as is common to ARDS (acute respiratory distress syndrome) or other ARI (acute respiratory infection), such as catechins. For example, Epigallocatechin Gallate or EGCG commonly found in Matcha or green tea has been reported to have anti-fibrosus benefits.

Compounds that inhibit of viral replication, such as theaflavin-3,3′-digallate (TF3), or black tea extract and/or Puer tea extracts, that has been found to be a 3CL^{Pro} inhibitor to inhibit viral replication similar to the mechanisms proposed for SARS-CoV-1.

Anti-inflammatory compounds, such as Hyaluranoic Acid blockers to reduce fluid uptake into the lungs. These may be included, for example, if the person is exhibiting strong host inflammatory response and the person is having trouble breathing. In some aspects, the person may be exhibiting elevated inflammatory markers, such as IL-6, CRP, LDH, Troponin, NT-proBNP, ferritin, D-dimer, and/or exhibiting sepsis, shock, ARDS, hypoxia, or cardiac failure.

Probiotic microbial strains that reduce or inhibit opportunistic pathogens, stimulate the immune system, and/or ameliorate gut dysbiosis, such as “leaky gut” issues whereby infectious corona virus may be crossing the intestinal cell wall barrier and into the bloodstream or other parts of the body. Examples of probiotic organisms that may be included, alone or in any combination, are set forth in Table 1.

TABLE 1
Probiotic Organisms.

| Probiotic | | |
|---|---|---|
| organism | Notes | Function |
| Bifidobacterium | Can be used individually | Helps maintain a healthy |
| lactis | or together in | respiratory function |
| | combination | Reinforcement of the natural |
| | | defenses |
| | | Reduction of the incidence, |
| | | severity and duration of Acute |
| | | Respiratory Infections (ARI) |
| | | during the cold season |
| Lactobacillus | Can be used individually | Reduce C. difficle, |
| rhamnosus | or together in | improve child respiratory IgA, |
| | combination | Improve post-pardum |
| | | depression, immunity, |
| | | reduce e. coli |
| Lactobacillus | Can be used individually | Reduce respiratory tract |
| fermentum | or together in | challenges |
| | combination | |
| Lactobacillus | Can be used individually | Reinforcement of the natural |
| plantarum | or together in | defenses |
| | combination | Reduction of the intestinal |
| | | discomfort |
| | | Rebalance of the intestinal |
| | | microbiota |
| | | Reduction of the inci-dence, |
| | | severity and duration of Acute |
| | | Respiratory Infections (ARI) |
| | | during the cold season |
| Bifidobacterium | Can be used individually | Reduce cytokine syndrome |
| breve | or together in | |
| | combination | |
| Lactobacillus | Can be used individually | Creates CO2 and lactic acid |
| brevis | or together in | during fermentation and |
| | combination | enhances natural killer cell |
| | | activity in the elderly |
| Lactococcus lactis | Can be used individually | The motilities of Pseudomonas, |
| | or together in | Vibrio and Leptospira strains |
| | combination: | were also severely disrupted by |
| | | lactose utilization by L. lactis |
| Bacillus coagulans | Can be used individually | Reduce pseudomonas or other |
| | or together in | opportunistic pathogens |
| | combination | |

As discussed herein, the invention provides the use of companion microbiome analysis information to identify opportunistic pathogens to indicate a dysbiosis or potential onset/recovery of respiratory issues and to optionally treat a patient with a customized therapy including a probiotic, pre-biotic or metabolite of the gut.

In some aspects, the present invention may be used to monitor treatment of a subject adminstered a therapeutic composition of the invention. For example, prior to treatment with a a therapeutic composition, such as a probiotic, a sample obtained from the digestive tract of a subject may be obtained and the genetic material of the microbes therein extracted as disclosed herein and subjected to metagenomics analysis. Then during and/or after treatment, a second sample may be obtained from the digestive tract of the subject and the genetic material of the microbes in the second sample extracted as disclosed herein and subjected to metagenomics analysis, the results of which are compared to the results of the metagenomics analysis of the first sample. Then, based on the comparative results, the treatment of the subject may be modified to obtain a desired population of microbes in the gut of the subject. For example, a therapeutic composition that includes a microbe whose amount is desired to be increased in the gut of the subject may be administered to the subject.

In some embodiments, the fecal sample may be mixed or cultured for determination of metabolomic of microbial fecal community. Metabolomic profile can then be used to determine probiotic strains that would benefit the individual. Examples of metabolomic profiles include those affecting energy metabolism, nutrient utilization, insulin resistance, adiposity, dyslipidemia, inflammation, short-chain fatty acids, organic acids, cytokines, neurotransmitters chemicals or phenotype and may include other metabolomic markers.

The method of the present invention is used to generate a customized therapeutic formulation and analyze the microbiome content in the gut of the subject. In one aspect, based on the microbiome content in the gut of the subject and any desired changes thereto, one may select one or more probiotics (optionally in combination with any other ingredient described herein) that contain the microbes that are desired to be increased and/or maintained in the subject's microbiome health. In one aspect, based on the microbiome content in the gut of the subject and any desired changes thereto, one may select one or more probiotics that contain the microbes that are desired to be increased and/or maintained in the subject's gut balance in relation to the macronutrient content they are getting from their food source as recorded by survey information from the individual directly or by the present invention of gut organism nucleic acid analysis.

Custom tailored probiotics may not be in equal amounts but are formulated based on relative abundance detected from the individual gut/fecal sample. These formulations are geared to modulate the microbiome to a healthy status. The healthy status of a microbiome is determined by the use of existing aggregate private and public databases such as metaHIT™, Human Microbiome Project™, American Gut Project™, and the like. The healthy status may also be determined individually when a person has no known issues and is in good health, from a blood biomarker checkup perspective, and then has their full microbiome profile completed. After one or several microbiome signatures have been completed then the average of some/all of the microbes found can be understood for that individual and variances from that average can be accessed to determine if they are in dysbiosis. Microbiome profiles can be aggregated into groups that are then assigned a barcode for rapid bioinformatic assignment. Groups can be created by single or multiple phenotypic, diagnostic, or demographic information related to the individual from which the sample was collected from. A unique group can be determined from another group by using statistical models such as linear distance calculations, diversity values, classifiers such as C4.5 decision tree, or principal component analysis an comparing to an aggregate known population such as “normals” defined by the Human Microbiome Project or American Gut Project.

Thus, in some embodiments, the present invention may be used to screen the gut microbiome of a given subject and then custom tailor a therapeutic regimen to the given subject based on the subject's gut microbiome and/or exposure to a pathogen.

In some embodiments, the present invention may be used to restore a subject's gut flora and/or fauna to homeostasis after an event that has caused a shift in the subject's microbiota from balanced microbiome to one that is causing or may be causing negative side effects, disorders, and/or disease. Health conditions can include infection, e.g., viral infection, or symptoms related thereto, such as respiratory complications and/or dysbiosis.

Thus, in some aspects, a ratio of a first given microbe to a second given microbe in the gut of a subject is determined using the methods described herein and then if the ratio is undesired or abnormal, the subject is administered a treatment to modify the ratio to be a desired ratio. In some embodiments, the amount of a first given microbe in a gut of a subject relative to the total amount of all the microbes in the gut of the subject is determined using the methods described herein and then if the relative amount of the first given microbe is undesired or abnormal, the subject is administered a treatment to modify the amount to be a desired amount. Re-testing of their gut microbiome maybe used to determine well they are adhering to the macronutrient and food guidance. Such treatments include administering to the subject: a probiotic containing one or more microbes whose amounts are desired to be increased in the gut of the subject, an antimicrobial agent, e.g., an antibiotic, an antifungal, an antiviral, or the like, to kill or slow the growth of a microbe or microbes whose amounts are desired to be decreased in the gut of the subject, a diet and/or a natural product or extract thereof, that supports the growth or maintenance of a healthy gut microbiome, e.g., a prebiotic, pland extract, metabolite, vitamin, enzyme, co-enzyme and the like.

Scoring of the microbiome signature overall uses a similar decision tree, algorithm, artificial intelligence, script, or logic tree as represented in Table 2. This system enables a score that helps a user understand how healthy their gut microbiome is and if they need to take action on a few or many challenges found. Challenges can include but not limited to, identification of known pathogenic organisms, count and identification of opportunistic pathogens, latent organisms known to cause pathogenic affects when given opportunity, lack of support for good microbial environment but their composition or lack of key strains, overall diversity and count of unique organisms found in top 10 and or organisms with greater than 0.1% prevalence.

Diversity cut offs were determined from an aggregate of sample analysis and a cutoff is determined at x relative abundance. For example, if x=0.1% then 352 unique organisms make up the average healthy profile. Then apply standard deviations around this number and using a Gaussian distribution and percentile under the curve analysis we can score how close to the average diversity number from our database average. The lower your diversity number and further away from the average you are then the less that microbiome would score. The higher the number and the greater your diversity is the more that microbiome would score. This type of scoring categories along with probiotic score will determine a number and visual metered score for the custom to understand how healthy their microbiome is. An example of the graphic visualization is included below. Where low is equal to low microbiome quality and high is equal to high microbiome quality and score. Low->30 out of 100, Med>65 out of 100, High=65 or greater out of 100.

An example of a scoring and probiotic formula algorithm is included in Table 2 below. Table 2 can be represented as decision tree, algorithm, artificial intelligence, script, or logic tree. The function of such decision tree, algorithm, artificial intelligence, script, or logic tree would be output a score of wellness of the individual microbiome as related to probiotics detected and to provide formulation and dosing recommendations for probiotic usage.

TABLE 2
Example Decision Table for Probiotic Scoring and Formulation.
Includes the Utilization of a Probiotic Strain Database, Metagenomic
Analysis Database, and Literature Curation Database.

| Criteria | | Criteria | |
|---|---|---|---|
| Number | Criteria | Answer | Score or Inclusion/Exclusion |
| 1 | Greater than 100 | Yes | If yes then include |
| | reads | | |
| 2 | Greater than 50% of | Yes | |
| | total probiotic reads | | |
| 3 | Greater than 10,000 | Yes | If yes do not include in probiotic formula |
| | reads | | |
| 4 | Greater than 50% of | No | |
| | total reads | | |
| 5 | Greater than 30,000 | Yes | If yes do not include in probiotic formula |
| | reads | | |
| 6 | Greater than 30,000 | Yes | If x > 5 then score + 20, x > 3 score 10, x > 1 |
| | reads for x number | | score 5 |
| | of probiotics | | |
| 7 | Total number of | x | If x > 10 then score + 20, x > 10 then score 10, |
| | microbes above 100 | | x > 5 score 5 |
| | reads (count) | | |
| 8 | Query for probiotic | Yes | Include in formula at 20 CFU/g or greater |
| | strains and output | | |
| | where 1 = yes and 4 | | |
| | is no and 6 is no and | | |
| | the number of reads | | |
| | is less than 1000 | | |
| 9 | If bacillus | Yes | Do not include |
| 10 | If lactobacillus | Yes | If x > 10000 score + 20, if x > 1000 score + 10, |
| | acidophilus greater | | if x > 100 score + 5 |
| | than x reads | | |
| 11 | If bacillus genus | Yes | If x > 1000 score + 20, if x > 100 score + 10, if x > |
| | greater than x reads | | 10 score + 5 |
| 12 | If Saccharomyes | Yes | If x > 1000 score + 20, if x > 100 score + 10, if x > |
| | boulardi greater than | | 10 score + 5 |
| | x reads | | |
| 13 | If infant if nursing | Yes | If x > 10 then score + 5, x > 30% then score + 10, |
| | and bifidobacterium | | x > 50% then score + 20, x > 70% then |
| | infantis > x % | | score + 30 |
| 14 | If not infant, not | Yes | If x > 20 then score + 5, if x > 10 then score + 10, |
| | child and | | if x < 10 then score + 20 |
| | bifidobacterium | | |
| | infantis > x % | | |

| | |
|---|---|
| 15 | Query to probiotic function, if function table is equal to health phenotype or |
| | healthDx then include in formula unless 3 or 5 = yes |

Additional examples of microbes that may be included in a therapeutic formulation of the invention are listed in Table 3.

TABLE 3
List of Strains of Gut Bacteria That Can be Used to Restore
Conditions and Profiles of the Microbial Ecosystems.

| Organism | Organism | Combination species | |
|---|---|---|---|
| Genus | species | it works with | Prebiotic or Postbiotics |
| Akkermansia | muciniphila | Methanobevibacter | Resistant starches, |
| | KLE1798, | smithii, | inulin, chicory root, |
| | KLE1797, | Faccalibacterium | oligosacharrides, soluble |
| | KLE1605 | prausnitzii Roseburia | fiber |
| | | hominis, Prevotella | |
| | | copri | |
| Faecalibacterium | prausnitzii strain | Bacteroides ovatus, | Frucoto-oligosaccharide, |
| | A2165, M21/2, | Bacteroides | inulin, other |
| | KLE1255, | uniformis, | oligosaccharides |
| | | Bacteroides caccae | |
| Methanobrevibacter | Smithii | Akkermansia | Resveratrol, green tea |
| | TS94C | muciniphila, | extract, other herbs, |
| | | Faecalbacterium | oligosaccharides, and |
| | | prausnitzii, | biochemicals |
| | | Roserburia hominis, | |
| | | Bacteroides uniformis | |
| Bifidobacterium | Pseudocatenulatum | Other probiotic strains | Natural herbs and |
| | IPLA36007, | just at B. animalis | extracts that increase |
| | DSM 20438, | lactis, B animalis | dopamine or serotonin as |
| | Catenulatum | infantis, | well as XOS that support |
| | DSM 16992 | Strephococcus | growth of |
| | | thermophilus and L. | Bifidobacterium |
| | | planantarum | |
| Bacteroides | xylanisolvens | Bacteroides ovatus, | Xylans, xylitol, plant |
| | CL03T12C04 | caccae, uniformis, | celluloses or other |
| | | xylanisolvens, | synthetic celluloses |
| | | Butyrivibrio crossotus | |
| Bacteroides | cellulitis | Bacteroides ovatus, | Plant celluloses or other |
| | | caccae, uniformis, | synthetic celluloses, |
| | | xylanisolvens, | plant based fibers, |
| | | Butyrivibrio crossotus | |
| Anaerostipes | Hadrus DSM | Facalibacterium | Walnuts, Resveratrol |
| | 3319 | prausnitzii, | |
| | | Saccharomyes | |
| | | boulardii | |
| Butyrivibrio | Crossotus DSM | Facalibacterium | Walnuts, Resveratrol |
| | 2876 | prausnitzii, | |
| | | Bacteroides ovatus, | |
| | | caccae, uniformis, | |
| | | cellulitis | |
| Gemmiger | formicilis | Facalibacterium | Walnuts |
| | | prausnitzii | Resveratrol |
| | | | Saccharomyces boulardii |
| Roseburia | faecis | Roseburia hominis + | Resistant starches, |
| | | Roseburia | dietary fiber, |
| | | intestinalis + Roseburia | Bifidobacterium |
| | | inulinivorans | pseudocatenulatum |
| | | | Catenulatum, Dopamine, |
| | | | Vitamin D, fungi, yeasts, |
| | | | algae, lichens, and |
| | | | plants, such as oats and |
| | | | barley. |
| Roseburia | Inulinivorans | Roseburia hominis + | Resistant starches, |
| | DSM 16841, | Roseburia | dietary fiber, |
| | | intestinalis + | Bifidobacterium |
| | | Roseburia faecis | pseudocatenulatum |
| | | | Catenulatum, Dopamine, |
| | | | Vitamin D, fungi, yeasts, |
| | | | algae, lichens, and |
| | | | plants, such as oats and |
| | | | barley. |
| Roseburia | Hominis A2183, | Roseburia | Resistant starches, |
| | | intestinalis + | dietary fiber, |
| | | Roseburia faecis + | Bifidobacterium |
| | | Roseburia | pseudocatenulatum |
| | | inulinivorans | Catenulatum, Dopamine, |
| | | | Vitamin D, fungi, yeasts, |
| | | | algae, lichens, and |
| | | | plants, such as oats and |
| | | | barley. |
| Roseburia | intestinalis | Roseburia hominis + | Resistant starches, |
| | | Roseburia | dietary fiber, |
| | | inulinivorans + | Bifidobacterium |
| | | Roseburia faecis | pseudocatenulatum |
| | | | Catenulatum, Dopamine, |
| | | | Vitamin D, fungi, yeasts, |
| | | | algae, lichens, and |
| | | | plants, such as oats and |
| | | | barley. |
| Bacteroides | uniformis | Bacteroides ovatus, | Fructo-oligosaccharides, |
| | dnLKV2, etc | caccae, | resistant starches, red |
| | | Facalibacterium | wine, L-citrulline |
| | | prausnitzii, Roseburia | |
| | | species, Prevotella | |
| | | copri, Eubacterium | |
| | | species | |
| Bacteroides | ovatus | Bacteroides | Fructo-oligosaccharides, |
| | | uniformis, caccae, | resistant starches, red |
| | | Facalibacterium | wine, L-citrulline |
| | | prausnitzii, Roseburia | |
| | | species, Prevotella | |
| | | copri, Eubacterium | |
| | | species | |
| Prevotella | copri | Bacteroides ovatus, | Saccharomyces |
| | | caccae, | organisms, legumes and |
| | | Facalibacterium | lentils, Mediterranean |
| | | prausnitzii, Roseburia | diet, flaxseed, |
| | | species, Eubacterium | |
| | | species, | |
| | | Saccharomyces | |
| | | organisms, | |
| Bifidobacterium | kashiwanohe nse | Bifidobacterium | Natural herbs and |
| | PV20-2, DSM | longumsp infantis, | extracts that increase |
| | 21854, JCM | Bifidobacterium | dopamine or serotonin as |
| | 15439 etc | | well as XOS that support |
| | | | growth of |
| | | | Bifidobacterium |
| Papillibacter | cinnamivoran s | Bacteroides cellulitis, | Cinnamon oils, shea |
| | DSM 12816, etc | xylanisolvens | butter, balsams |
| Lactobacillus | ruminis | Lactobacillus | Oligo-saccharides |
| | ATCC 25644, | acidophilus, | (mono-, di-, tri-, and |
| | L5, S21, S23, | plantarum, reuteri, | tetra-) and other |
| | S36, S38, etc | delbrueckii, and other | oligosaccharides, lactose, |
| | | organism that have | lactulose, GOS inulin or |
| | | been shown to reduce | other inulins |
| | | gut microbes that | |
| | | have negative impacts | |
| Oxalobacter | formigenes | Lactobacillus | Calcium, Magnesium, |
| | HOxBLS, etc | acidophilus, | Vitamins (like vitamin |
| | | Oxalobacter | C) |
| | | vibrioformis | |
| Bacteroides | caccae | Bacteroides | Fructo-oligosaccharides, |
| | CL03T12C61, | uniformis, ovatus, | resistant starches, red |
| | ATCC 43185 etc | Facalibacterium | wine, L-citrulline |
| | | prausnitzii, Roseburia | |
| | | species, Prevotella | |
| | | copri, Eubacterium | |
| | | species | |
| Eubacterium | rectale ATCC | Eubacterium siraeum, | Inulin, dietary fibers, |
| | 33656, etc | ramulus, eligens, | resistant starches, raw |
| | | hallii, Akkermansia | banana extract, |
| | | muciniphilia | |
| Eubacterium | siraeum DSM | Eubacterium rectale, | Inulin, dietary fibers, |
| | 15702 | ramulus, eligens, | resistant starches |
| | | hallii, Akkermansia | |
| | | muciniphilia | |
| Eubacterium | eligens ATCC | Eubacterium rectale, | Inulin, dietary fibers, |
| | 27750 | ramulus, siraeum, | resistant starches |
| | | hallii, Akkermansia | |
| | | muciniphilia | |
| Eubacterium | hallii DSM 3353 | Eubacterium rectale, | Inulin, dietary fibers, |
| | | ramulus, siraeum, | resistant starches |
| | | eligens, Akkermansia | |
| | | muciniphilia | |

It has been suggested that, after discharge from a hospital, some patients remain/return viral positive and others even relapse As patients of a pandemic, such as COVID-19, return to the workforce, to prevent large numbers of re-infection, effective screening of the population is necessary to ensure immunity to resilience to viral infection by viruses such as SARS-CoV-2. Recent data suggests that over 50% of cases of COVID-19 indicate an issue with their gut. The onset of COVID-19 may begin in the gut and not show any respiratory symptoms until later. This infection has been shown to contain live virus which may be transmittable via fecal matter or orally and is a significant risk for service workers returning to work.

As such, the invention further provides a method for screening a subject for exposure to an infectious pathogen and treating the subject where the subject has been exposed to the infectious pathogen and/or exhibits symptoms associated with pathogenic infection. In some aspects, the present disclosure provides the following methodology for managing COVID-19 pandemic and return to the workforce in consideration for people that may present with gastrointestinal issues or can be used more broadly for all cases of screening.

In some aspects, the method includes screening a screening a subject for a previous exposure to a virus using an antibody assay, and where the antibody assay is negative, screening the subject for the virus using a PCR based assay and administering the subject a therapeutic composition of the invention.

In some aspects, the method includes screening a subject for a previous exposure to a virus using an IgG/IgM specific antibody assay, wherein if the subject is IgM negative, the subject is screened for the virus via a PCR based assay and administered the therapeutic composition of the invention where the PCR based assay is positive and then rescreened using the IgG/IgM specific antibody assay after about 3 to 21 days, and wherein if the subject is IgM positive, the subject is administered the therapeutic composition of therapeutic composition of the invention and then rescreened using the IgG/IgM specific antibody assay after about 3 to 21 days.

In some aspects, the method includes screening a subject for a viral infection using a PCR based assay, wherein if the PCR based assay is positive the subject is administered the therapeutic composition of any one of claims 22 to 35 and then rescreened using the PCR based assay after about 3 to 21 days, and wherein if the PCR based assay is negative, the subject is screened for a previous exposure to the virus using an IgG/IgM specific antibody assay, and wherein if the subject is IgM negative, the subject is screened for risk of infecting another subject via a PCR based test and administered the therapeutic composition of the invention where the PCR based assay is positive and then rescreened using the IgG/IgM specific antibody assay after about 3 to 12 days, and wherein if the subject is IgM positive, the subject is administered the therapeutic composition of the invention and then rescreened using the IgG/IgM specific antibody assay after about 3 to 21 days.

The following presents schemas for screening and treatment in some aspects of the invention.

Screen via the IgG and IgM test for antibodies.

A) If IgG positive or negative AND IgM negative, then screen via the RT-qPCR assay of Example 1 to test for viral shedding risk or longer term of infectivity risk

i) If RT-qPCR stool is negative, then return to work

ii) If RT-qPCR stool is positive, then begin natural product described above to reduce viral load and stay home and retest of IgG/IgM test in 6-10 days

b) If IgM positive then begin administration of therapeutic formulation of the invention to strengthen immune system along with other standard of care procedures and quarantine for 14-21 days and retest back to step A.

Screen via detection method of Example 1 or other available RT-qPCR nasal swab test that uses a stabilizer at collection (Whatman-like paper) to stabilize RNA and put through extraction and analysis process.

A) If positive, then begin probiotics described above to reduce viral load and stay home and retest in 3-10 days
B) If negative, then reflex to IgG/IgM specific antibody test to determine previous infection and immunity

i) If IgG positive or negative AND IgM negative, then screen via our stool RT-qPCR to test for viral shedding risk or longer term of infectivity risk
(1) If RT-qPCR stool is negative, then return to workforce
(2) If RT-qPCR stool is positive, then begin administration of therapeutic formulation of the invention to reduce viral load and stay home and retest of IgG/IgM test in 6-10 days

ii) If IgM positive then begin administration of therapeutic formulation of the invention to strengthen immune system along with other standard of care procedures and quarantine for 14-21 days and retest back to step A or B.

Screening by IgG and IgM antibody test and nasal RT-qPCR for screening to return to workforce then use stool RT-qPCR for determining eligibility for these tests.

A) If positive, then reflex to nasal RT-qPCR test in step 2 and quarantine and begin natural product described here
b) If negative, then reflex to IgG and IgM antibody test in step 1 with higher likelihood you can return to workforce

In various aspects, treatment may include administration of a therapeutic formulation of the invention to a subject. As discussed herein, administration may be combined with various different treatment modalities. Examples of such treatments are included, but not limited to those set forth in Table 4.

TABLE 4
Conventional treatment of patients with SARS-CoV-2 infection.

| Type of treatment | Therapeutic agent or device |
|---|---|
| Oxygen therapy | Nasal cannula |
| | Non-invasive mechanical ventilation |
| | Invasive mechanical ventilation |
| | ECMO* |
| Antibiotics | Amoxicillin |
| combination | Azithromycin |
| | Fluoroquinolones |
| Antivirals | Lopinavir/ritonavir |
| | Ribavirin |
| | Favipiravir (T-705) |
| | Remdesivir |
| | Oseltamivir |
| | Chloroquine |
| | Interferon |
| Corticosteroids | Methylprednisolone |
| Convalescent plasma | Convalescent plasma |

In various aspects, the invention utilizes a PCR assay, such as an RT-qPCR assay as set forth in Example 1, for detection of SARS-CoV-2 in a biological sample.

As such, the invention provides a method for detecting SARS-CoV-2 in a biological sample, such as a stool sample. In some aspects, the method includes:
obtaining a biological sample comprising ribonucleic acids;
reverse transcribing the ribonucleic acids to obtain cDNA;
contacting the cDNA with a first and/or second primer set, and a DNA polymerase to produce a first and/or second PCR product, wherein the first primer set comprises SEQ ID NOs: 1 and 2 and the second primer set comprises SEQ ID NOs: 5 and 6;
hybridizing to the first PCR product a first nucleic acid probe comprising SEQ ID NO: 3 and/or SEQ ID NO: 4, and/or hybridizing to the second PCR product a second nucleic acid probe comprising SEQ IN NO: 7 and/or 8; and
detecting hybridization of the first nucleic acid probe to the first PCR product and/or detecting hybridization of the second nucleic acid probe to the second PCR product,
wherein hybridization of the first nucleic acid probe to the first PCR product, hybridization of the second nucleic acid probe to the second PCR product, is indicative of the presence of SARS-CoV-2 nucleic acids in the biological sample.

In some aspects, method includes:
contacting the cDNA with a control primer set, and a DNA polymerase to produce a control PCR product, wherein the control primer set comprises SEQ ID NOs: 9 and 10;
hybridizing to the control PCR product a control nucleic acid probe comprising SEQ ID NO: 11 and/or SEQ ID NO: 12; and
detecting hybridization of the control nucleic acid probe to the control PCR product.

In another embodiment, the invention provides a kit for detecting SARS-CoV-2. The kit includes first and/or second primer set, wherein the first primer set comprises SEQ ID NOs: 1 and 2 and the second primer set comprises SEQ ID NOs: 5 and 6, a first nucleic acid probe comprising SEQ ID NO: 3 and/or SEQ ID NO: 4, and/or a second nucleic acid probe comprising SEQ IN NO: 7 and/or 8; and optionally reagents for conducting a reverse transcription-polymerase chain reaction using a) and b). In some aspects, the kit further includes a control primer set, wherein the control primer set comprises SEQ ID NOs: 9 and 10, and a control nucleic acid probe comprising SEQ ID NO: 11 and/or SEQ ID NO: 12.

Kits of this invention include all the reagents to perform a PCR reaction wherein each of the labeled probes of the kit are used to monitor a sample for the presence, absence or quantity of SARS-CoV-2. In various aspects, one or more of the oligonucleotides of the kit perform as the primers in the PCR reaction.

A typical kit will contain at least two primers (e.g., SEQ ID NOs: 1 and 2, and/or SEQ ID NOs: 5 and 6), at least one probe (e.g., SEQ ID NOs: 3 and 4, and/or SEQ ID NOs: 7 and 8), nucleotide triphosphates, polymerase enzyme (preferably thermostable polymerase) and a buffer solution (with controlled ionic strength, controlled magnesium content and pH modulator).

As used herein the term “amplified” or “amplification” refers to the production of many DNA copies from one or a few copies.

As used herein the term “multiplex PCR” refers to PCR, which involves adding more than one set of PCR primers to the reaction in order to target multiple locations throughout the genome; it is useful for DNA typing because, inter alia, the probability of identical alleles in two individuals decreases with an increase in the number of polymorphic loci examined. Furthermore, multiplexing with an internal control (e.g., human RNase P) provides internal control of the whole PCR without affecting sensitivity or specificity of the SARS-CoV-2 real-time PCR.

As used herein, a DNA segment is referred to as “operably linked” or “operatively linked” when it is placed into a functional relationship with another DNA segment. Generally, DNA sequences that are operably linked are contiguous, and in the case of a signal sequence or fusion protein both contiguous and in reading phase. However, enhancers need not be contiguous with the coding sequences whose transcription they control. Linking, in this context, is accomplished by ligation at convenient restriction sites or at adapters or linkers inserted in lieu thereof.

As used herein, “PCR” generally refers to a method for amplifying a DNA or RNA base sequence using a heat-stable polymerase and two oligonucleotide primers, one complementary to the (+)-strand at one end of the sequence to be amplified and the other complementary to the (−)-strand at the other end. Because the newly synthesized DNA or cDNA strands can subsequently serve as additional templates for the same primer sequences, successive rounds of primer annealing, strand elongation, and dissociation produce rapid and highly specific amplification of the desired sequence.

As used herein, the term “probes” refer to nucleic acid sequences of variable length, preferably between at least about 10 nt or about 100 nt depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies, preferably PCR, more preferably RT-PCR, and even more preferably in real-time RT-PCR.

As used herein, the term “primer” refers to a short, artificial oligonucleotide strands usually not more than fifty, preferably 18-25 bp nucleotides (since DNA is usually double-stranded, its length is measured in base pairs; the length of single-stranded DNA is measured in bases or nucleotides) that exactly match the beginning and end of the genomic fragment to be amplified. Primers anneal (adhere) to the DNA template at the starting and ending points, where the DNA-Polymerase binds and begins the synthesis of the new DNA strand. The choice of the length of the primers and their melting temperature (Tm) depends on a number of considerations. The melting temperature of a primer—not to be confused with the melting temperature of the DNA in the first step of the PCR process—is defined as the temperature below which the primer will anneal to the DNA template and above which the primer will dissociate (break apart) from the DNA template. The melting temperature increases with the length of the primer. Primers that are too short would anneal at several positions on a long DNA template, which would result in non-specific copies. On the other hand, the length of a primer is limited by the temperature required to melt it. Melting temperatures that are too high, (e.g., above 80° C.), can also cause problems since the DNA-Polymerase is less active at such temperatures. The optimum melting temperature is between 60° C. and 75° C. A forward sequencing primer anneals 5′ with respect to the reverse primer, and the reverse sequencing primer that anneals 3′ with respect to the forward primer. The relationship between the primers and the reference sequence depends on the coordinate system that is used. The forward primer's annealing positions will usually be less than the annealing positions of the reverse primer since the forward primer should fall to the logical left of the reverse primer in the coordinate system.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide. Stringent conditions are known to those skilled in the art and can be found in Ausubel et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65.degree. C., followed by one or more washes in 0.2×SSC, 0.01% BSA at 50° C.

As used herein, the term “TaqMan” generally refers to the probe used to detect specific sequences in PCR products by employing the 5′->3′ exonuclease activity of Taq DNA polymerase. The TaqMan probe (about 20-30 bp), disabled from extension at the 3′ end, consists of a site-specific sequence labeled with a fluorescent reporter dye and a fluorescent quencher dye. During PCR the TaqMan probe hybridizes to its complementary single strand DNA sequence within the PCR target. When amplification occurs the TaqMan probe is degraded due to the 5′->3′ exonuclease activity of Taq DNA polymerase, thereby separating the quencher from the reporter during extension. Due to the release of the quenching effect on the reporter, the fluorescence intensity of the reporter dye increases. During the entire amplification process this light emission increases exponentially, the final level being measured by spectrophotometry after termination of the PCR. Because increase of the fluorescence intensity of the reporter dye is only achieved when probe hybridization and amplification of the target sequence has occurred, the TaqMan assay offers a sensitive method to determine the presence or absence of specific sequences. Therefore, this technique is particularly useful in diagnostic applications, such as the screening of samples for the presence or incorporation of favorable traits and the detection of pathogens and diseases. The TaqMan assay allows high sample throughput because no gel-electrophoresis is required for detection. TaqMan probes depend on the 5′-nuclease activity of the DNA polymerase used for PCR to hydrolyze an oligonucleotide that is hybridized to the target amplicon. In particular, TaqMan probes are oligonucleotides that have a fluorescent reporter dye attached to the 5′ end and a quencher moeity coupled to the 3′ end. These probes are designed to hybridize to an internal region of a PCR product. In the unhybridized state, the proximity of the fluorescent reporter and the quench molecules prevents the detection of fluorescent signal from the probe. During PCR, when the polymerase replicates a template on which a TaqMan probe is bound, the 5′-nuclease activity of the polymerase cleaves the probe. This decouples the fluorescent and quenching dyes and the Fluorescence Resonance Energy Transfer (FRET) no longer occurs. Thus, fluorescence increases in each cycle, proportional to the amount of probe cleavage.

As used herein, the term “thermostable polymerase enzyme” refers to an enzyme, which is stable to heat and is heat resistant and catalyzes (facilitates) combination of the nucleotides in the proper manner to form the primer extension products that are complementary to each nucleic acid strand. Generally, the synthesis will be initiated at the 3′ end of primer and will proceed in the 5′ direction along the template strand, until synthesis terminates, producing molecules of different lengths. There may be a thermostable enzyme, however, which initiates synthesis at the 5′ end and proceeds in the other direction, using the same process as described above. The preferred thermostable enzyme herein is a DNA polymerase isolated from Thermus aquaticus. Various strains thereof are available from the Americal Type Culture Collection, Rockville, Md., and are described by T. D. Brock, J. Bact. (1969) 98:289-297, and by T. Oshima, Arch. Mircobiol. (1978) 117:189-196. One of these preferred strains is strain YT-1.

The real time RT-PCR method of the present invention allows infected humans with no clinical signs of SARS-CoV-2 to be detected. The standardized PCR system can be used as a robust tool for the highly sensitive and specific detection of SARS-CoV-2 in eradication campaigns or in case of emergencies.

In some aspects of this invention, a multiplex hybridization assay is performed. Multiplex analysis relies on the ability to sort sample components or the data associated therewith, during or after the assay is completed. In preferred embodiments of the invention, distinct independently detectable moieties are used to label component of two or more different complexes. The ability to differentiate between and/or quantitate each of the independently detectable moieties provides the means to multiplex a hybridization assay because the data which correlates with the hybridization of each of the distinctly (independently) labeled complexes to a target sequence can be correlated with the presence, absence or quantity of each target sequence or target molecule sought to be detected in a sample.

Consequently, the multiplex assays of this invention may be used to simultaneously detect the presence, absence or quantity of two or more target sequence or target molecule in the same sample and in the same assay. Because the complexes are self-indicating, and can be designed to be independently detectable, the multiplex assays of this invention can be performed in a closed tube format to provide data for simultaneous real-time and end-point analysis of a sample for two or more target sequences or target molecules of interest in the same assay. Additionally, the assays can be further multiplexed by the incorporation of unimolecular probes to thereby confirm assay performance or be used to identify a specific feature of a target sequence or target molecule of interest.

As illustrated by the examples that follow, the oligonucleotides of the invention are particularly useful for applications involving multiple oligonucleotides sets wherein each oligonucleotide contains at least one independently detectable moiety. Preferably, the independently detectable moieties are independently detectable fluorophores. For example, a mixture of one or more different oligonucleotides may be used to detect each of four different target sequences, wherein one or more oligonucleotides comprises one or more independently detectable fluorophores. For this example, detection of the presence, absence or quantity of the different target sequences is made possible by the detection and/or quantitation of each of the different independently detectable fluorophores after the mixture has been incubated with the sample of interest. As previously discussed, the oligonucleotides may also be used in assays wherein the independently detectable moieties are used to distinguish the operation of the same or different processes occurring in the same assay. Such multiplex assays are possible whether the oligonucleotides are used as probes or as primers.

In another embodiment of the invention, the probes of the invention are oligonucleotide probes. In some aspects the probes comprise up to 50 nucleotides, preferably the probe is about 10-30 nucleotides long, and more preferably oligonucleotide probe is about 15-25 nucleotides long. In some aspects, the probe is of sequence SEQ ID NO: 3, 4, 7 or 8. In some aspects, the probe is fluorescently labeled.

The labels attached to the probes of this invention comprise a set of energy or electron transfer moieties comprising at least one donor and at least one acceptor moiety. The label can be any type of differentiating label (e.g., a nucleic acid sequence that is not CSF-specific), a detectable molecule (e.g., a fluorescent group that can be inserted by known methods using, for example, fluorescein isothiocyanate), or digoxigenin, or a molecule that can be immobilized, such as biotin (by means of which the oligonucleotide can be bound to a streptavidin-coated surface, for instance).

Typically, the label will include a single donor moiety and a single acceptor moiety. Nevertheless, a label may contain more than one donor moiety and/or more than one acceptor moiety. For example, a set could comprise three moieties. Moiety one may be a donor fluorophore which, when exited and located in close proximity to moiety two, can then transfer energy to moiety two of the label. Thereafter, moiety two, which when excited and located in close proximity to moiety three, can transfer energy to moiety three of the label. Consequently, energy is transferred between all three moieties. In this set, moiety two is both an acceptor of energy from moiety one and a donor of energy to moiety three.

The donor and acceptor moieties operate such that one or more acceptor moieties accepts energy transferred from the one or more donor moieties or otherwise quench signal from the donor moiety or moieties. Transfer of energy may occur through collision of the closely associated moieties of a label (non-FRET) or through a nonradiative process such as fluorescence resonance energy transfer (FRET). For FRET to occur, transfer of energy between donor and acceptor moieties requires that the moieties be close in space and that the emission spectrum of a donor have substantial overlap with the absorption spectrum of the acceptor (See: Yaron et al. Analytical Biochemistry, 95, 228-235 (1979) and particularly page 232, col. 1 through page 234, col. 1). Alternatively, non-FRET energy transfer may occur between very closely associated donor and acceptor moieties whether or not the emission spectrum of a donor moiety has a substantial overlap with the absorption spectrum of the acceptor (See: Yaron et al. Analytical Biochemistry, 95, 228-235 (1979) and particularly page 229, col. 1 through page 232, col. 1). This process is referred to as intramolecular collision since it is believed that quenching is caused by the direct contact of the donor and acceptor moieties.

Preferred donor and acceptor moieties are fluorophore and quencher combinations, respectively. Numerous amine reactive labeling reagents are commercially available (as for example from Molecular Probes, Eugene, Oreg.). Preferred labeling reagents will be supplied as carboxylic acids or as the N-hydroxysuccinidyl esters of carboxylic acids. Preferred fluorochromes (fluorophores) include 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine 2, 3, 3.5, 5 and 5.5 are available as NHS esters from Amersham, Arlington Heights, Ill.) or the Alexa dye series (Molecular Probes, Eugene, Oreg.). The most preferred fluorophores are the derivatives of fluorescein and particularly 5 and 6-carboxyfluorescein. The acceptor moiety may be a second fluorophore but preferably the acceptor moiety is a quencher moiety. A quencher moiety is a moiety which can quench detectable signal from a donor moiety such as a fluorophore. Most preferably, the quencher moiety is an aromatic or heteroaromatic moiety which is substituted with one or more azo or nitro groups. The most preferred quencher moiety is 4-((−4-(dimethylamino)phenyl)azo)benzoic acid (dabcyl).

Methods for data analysis according to various aspects of the present invention may be implemented in any suitable manner, for example using a computer program operating on the computer system. An exemplary analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The analysis system according to various aspects of the present invention and its various elements provide functions and operations to facilitate microbiome analysis, such as data gathering, processing, analysis, reporting and/or diagnosis. The present analysis system maintains information relating to microbiomes and samples and facilitates analysis and/or diagnosis. For example, in the present embodiment, the computer system executes the computer program, which may receive, store, search, analyze, and report information relating to the microbiome. The computer program may comprise multiple modules performing various functions or operations, such as a processing module for processing raw data and generating supplemental data and an analysis module for analyzing raw data and supplemental data to generate a models and/or predictions.

The analysis system may also provide various additional modules and/or individual functions. For example, the analysis system may also include a reporting function, for example to provide information relating to the processing and analysis functions. The analysis system may also provide various administrative and management functions, such as controlling access and performing other administrative functions.

The use of the singular can include the plural unless specifically stated otherwise. As used in the specification and the appended claims, the singular forms “a”, “an”, and “the” can include plural referents unless the context clearly dictates otherwise. The use of “or” can mean “and/or” unless stated otherwise. As used herein, “and/or” means “and” or “or”. For example, “A and/or B” means “A, B, or both A and B” and “A, B, C, and/or D” means “A, B, C, D, or a combination thereof” and said “combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

The present invention is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions. In addition, although the invention is described in the medical diagnosis context, the present invention may be practiced in conjunction with any number of applications, environments and data analyses; the systems described herein are merely exemplary applications for the invention.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### RT-qPCR Assay for Detection of SARS-CoV-2

This example describes development and use of a RT-qPCR assay for use with the method of the invention.

Methodology

The laboratory developed (LDT) real-time RT-qPCR test on stool samples described herein is intended for the qualitative detection of nucleic acid from the SARS-CoV-2. The assay is a real-time reverse transcription polymerase chain reaction (rRT-PCR) test. The 2019-nCoV primer and probe set(s) is designed to detect nucleic acid (RNA) from SARS-CoV-2 in patient stool samples as recommended for testing by public health authority guidelines.

The oligonucleotide primers and probes for detection of SARS-CoV-2 were designed specifically to detect regions of the virus nucleocapsid (N) gene. Two primer/probe sets are specific for 2 regions of the N gene of SARS-CoV-2, as well as a primer/probe set to detect the human RNase P gene (RP) in control samples and clinical specimens.

Participant samples were randomly selected from our stool sample repository, by carefully sorting the samples with a collection date that was after the outbreak of the 2019-2020 SARS-CoV-2 pandemic. RNA was extracted and purified from stool samples using the assay described here. RNA was subjected to reverse transcription to form cDNA and subsequently amplified in the Applied Biosystems StepOnePlus Real-Time PCR™ Instrument with SDS version 2.3 software. The Taqman™ probe anneals to the specific target sequence located between the forward and reverse primers. After the generation of cDNA, during the extension phase of the PCR cycle, the 5′ nuclease activity of Taq polymerase degrades the probe, causing the reporter dye to separate from the quencher dye, generating a fluorescent signal. With each cycle, additional reporter dye molecules are cleaved from their respective probes, increasing the fluorescence intensity. Fluorescence intensity is monitored at each PCR cycle by Applied Biosystems StepOnePlus Real-Time PCR™ System with SDS version 2.3 software.

TABLE 5
Assay Master mix and Reagents.

| Reagents, Controls and | | |
|---|---|---|
| Primers | Targets | Description |
| TaqPath ™ 1-Step RT-qPCR | Reaction Master mix with Enzyme | Designed to use a single |
| Master Mix, CG | | protocol to assay both |
| (ThermoFisher) | | types of nucleic acid. |
| Assay negative control | None | No Template Control (NTC) |
| 2019-nCoV_N_Positive | N1 and N2 | plasmids contain the |
| Control (IDT) | | complete nucleocapsid(N) |
| (SEQ ID NOs: 1-8) | | gene from SARS_CoV-2 and |
| | | amplifies both N1 and N2 |
| Hs_RPP30 Positive Control | RP | The Hs_RPP30 Control |
| (IDT) | | contains a portion of the |
| (SEQ ID NOs: 9-12) | | RPP30 gene, a single copy |
| | | gene present in the human |
| | | genome. |

Primers

| | SEQ | | | | |
|---|---|---|---|---|---|
| | ID | | | | Final |
| Name | NO. | Description | Oligonucleotide Sequence (5′>3′) | Label | conc. |
| 2019- | 1 | 2019-nCoV_N1 | GAC CCC AAA ATC AGC GAA AT | None | 500 nM |
| nCoV_N1-F | | Forward Primer | | | |
| 2019- | 2 | 2019-nCoV_N1 | TCT GGT TAC TGC CAG TTG AAT | None | 500 nM |
| nCoV_N1-R | | Reverse Primer | CTG | | |
| 2019- | 3 | 2019-nCoV_N1 | FAM-ACC CCG CAT TAC GTT TGG | FAM, | 125 nM |
| nCoV_N1-P | | Probe | TGG ACC-BHQ1 | BHQ-1 | |
| 2019- | 4 | 2019-nCoV_N1 | FAM-ACC CCG CAT /ZEN/ TAC | FAM, | 125 nM |
| nCoV_N1-P | | Probe | GTT TGG TGG ACC-3IABkFQ | ZEN, | |
| | | | | 3IABkFQ | |
| 2019- | 5 | 2019-nCoV_N2 | TTA CAA ACA TTG GCC GCA AA | None | 500 nM |
| nCoV_N2-F | | Forward Primer | | | |
| 2019- | 6 | 2019-nCoV_N2 | GCG CGA CAT TCC GAA GAA | None | 500 nM |
| nCoV_N2-R | | Reverse Primer | | | |
| 2019- | 7 | 2019-nCoV_N2 | FAM-ACA ATT TGC CCC CAG CGC | FAM, | 125 nM |
| nCoV_N2-P | | Probe | TTC AG-BHQ1 | BHQ-1 | |
| 2019- | 8 | 2019-nCoV_N2 | FAM-ACA ATT TGC /ZEN/ CCC | FAM, | 125 nM |
| nCoV_N2-P | | Probe | CAG CGC TTC AG-3IABkF | ZEN, | |
| | | | | 3IABkFQ | |
| RP-F | 9 | RNAse P Forward | AGA TTT GGA CCT GCG AGC G | None | 500 nM |
| | | Primer | | | |
| RP-R | 10 | RNAse P Reverse | GAG CGG CTG TCT CCA CAA GT | None | 500 nM |
| | | Primer | | | |
| RP-P | 11 | RNAse P Probe | FAM - TTC TGA CCT GAA GGC | FAM, | 125 nM |
| | | | TCT GCG CG - BHQ-1 | BHQ-1 | |
| RP-P | 12 | RNAse P Probe | FAM-TTC TGA CCT /ZEN/ GAA | FAM, | 125 nM |
| | | | GGC TCT GCG CG-3IABkFQ | ZEN, | |
| | | | | 3IABkFQ | |

| RNA extraction controls | Targets | Description |
|---|---|---|
| Positive Extraction control | N1 and N2 | Extraction control included |
| SARS_COV-2 | | in every batch of extraction |
| Human genomic DNA | RP | Extraction control included |
| | | in every batch of extraction |
| Assay Reagents | Volume used | Total volume |
| TaqPath ™ 1-Step RT-qPCR | 5 ul | 20 ul |
| Master Mix, CG | | |
| (ThermoFisher) | | |
| Ultrapure H2O | 8.5 ul | |
| Primers/probes set | 1.5 ul | |
| Template | 5 ul | |

Results

The results showed positive cases in several participants as listed below. Four out of 133 stool (3%) specimens tested positive to COVID-19. The SARS-CoV-2 real-time RT-PCR assay was validated to show analytical sensitivity, specificity and high accuracy in detecting nucleic acids in stool samples.

Several studies have shown that the viral RNA remained positive in feces, even after test results for viral RNA in the respiratory tract converted to negative, indicating that the viral gastrointestinal infection and potential fecal-oral transmission can last even after viral clearance in the respiratory tract. This is due to the fact that Viral loads from stool samples were found to peak later in the disease, generally 2-3 weeks after symptom onset. This test will benefit the participants with ‘Covid-19 long haulers’, the group that experiences long-lasting coronavirus disease 2019 (COVID-19) symptoms. Asymptomatic participants do not exhibit any symptoms associated with COVID while they harbour the virus and yet are carriers and can spread the virus unknowingly. The assay will be aid in detecting the asymptomatic category of participants too.

TABLE 6
Samples detected with SARS-CoV-2 RT-qPCR assay.

| Sample ID of | | |
|---|---|---|
| Positive samples | Symptoms reported | Microbes detected |
| FC000028 | constipation | |
| SG004941 | Frequent diarrhoea (2-4 | High Clostridium sps. |
| | times/day) | |
| SG004942 | No symptoms | Low Faecalibacterium sps. |
| | | high clostridium and high |
| | | Alistipes |
| SG005020 | Bowel Movements too | High clostridium and high |
| | Frequent | Alistipes |

TABLE 7
Microbial abundance in SARS-CoV-2 positive sample SG004941.

| Microbes | Relative abundances |
|---|---|
| Faecalibacterium prausnitzii | 10.061 |
| Fusicatenibacter saccharivorans | 5.873 |
| [Eubacterium] eligens | 5.484 |
| Blautia wexlerae | 4.097 |
| Clostridioides difficile | 4.029 |
| Bacteroides uniformis | 3.349 |
| Anaerostipes hadrus | 3.328 |
| Clostridiales bacterium KLE1615 | 2.569 |
| Bacteroides dorei | 2.327 |
| [Eubacterium] hallii | 2.127 |
| Ruminococcus lactaris | 1.71 |
| Blautia obeum | 1.554 |
| Negativibacillus massiliensis | 1.486 |
| Blautia coccoides | 1.466 |
| Roseburia intestinalis | 1.429 |
| Ruminococcus bicirculans | 1.252 |
| Bacteroides sp. 3_1_40A | 1.164 |
| Roseburia faecis | 1.121 |
| Dorea formicigenerans | 0.966 |
| [Clostridium] bolteae | 0.956 |
| [Eubacterium] rectale | 0.945 |
| Bacteroides fragilis | 0.869 |
| [Clostridium] clostridioforme | 0.809 |
| Roseburia inulinivorans | 0.796 |
| Lachnospiraceae bacterium | 0.766 |
| 8_1_57FAA | |
| Intestinimonas butyriciproducens | 0.746 |
| Collinsella aerofaciens | 0.686 |
| Fusicatenibacter sp. | 0.588 |
| 2789STDY5834925 | |
| Bacteroides cellulosilyticus | 0.574 |
| bacterium LF-3 | 0.536 |
| Eubacterium ramulus | 0.522 |
| Oscillibacter sp. ER4 | 0.518 |
| Bacteroides vulgatus | 0.515 |
| [Ruminococcus] gnavus | 0.498 |
| Ruminococcus sp. 5_1_39BFAA | 0.491 |
| Clostridium sp. AT4 | 0.466 |
| Bacteroides intestinalis | 0.451 |
| Fournierella massiliensis | 0.449 |
| Bilophila wadsworthia | 0.42 |
| Bacteroides faecis | 0.399 |
| Coprococcus eutactus | 0.398 |
| Bifidobacterium longum | 0.364 |
| Dorea longicatena | 0.362 |
| Bacteroides ovatus | 0.357 |
| Bariatricus massiliensis | 0.345 |
| Clostridium sp. HMSC19A11 | 0.335 |
| Lachnospiraceae bacterium | 0.323 |
| 5_1_63FAA | |
| Anaerotruncus colihominis | 0.317 |
| Roseburia hominis | 0.306 |
| Butyricicoccus desmolans | 0.289 |
| Blautia sp. Marseille-P2398 | 0.288 |
| Clostridiales bacterium VE202-01 | 0.288 |
| Akkermansia sp. KLE1798 | 0.28 |
| Coprococcus comes | 0.28 |
| Lachnospira pectinoschiza | 0.272 |
| Clostridium sp. SS2/1 | 0.264 |
| Ruminococcus sp. JC304 | 0.259 |
| Barnesiella intestinihominis | 0.258 |
| Sutterella wadsworthensis | 0.257 |
| Roseburia sp. 831b | 0.255 |
| Blautia hydrogenotrophica | 0.253 |
| [Clostridium] leptum | 0.25 |
| [Ruminococcus] torques | 0.242 |
| Bacteroides salyersiae | 0.236 |
| Bacteroides thetaiotaomicron | 0.215 |
| Gemmiger formicilis | 0.21 |
| Sutterella sp. KLE1602 | 0.209 |
| Hungatella hathewayi | 0.207 |
| Blautia sp. Marseille-P3087 | 0.205 |
| Parabacteroides distasonis | 0.204 |
| [Bacteroides] pectinophilus | 0.196 |
| Bacteroides stercoris | 0.194 |
| Bacteroides finegoldii | 0.19 |
| Holdemania filiformis | 0.184 |
| Eisenbergiella tayi | 0.18 |
| [Clostridium] saccharolyticum | 0.179 |
| Akkermansia sp. KLE1797 | 0.177 |
| Subdoligranulum sp. 4_3_54A2FAA | 0.175 |
| Clostridium phoceensis | 0.174 |
| Clostridium sp. M62/1 | 0.172 |
| Roseburia sp. 499 | 0.168 |
| Dorea sp. AGR2135 | 0.167 |
| Flavonifractor plautii | 0.165 |
| Enterococcus faecium | 0.155 |
| Clostridiales bacterium 1_7_47FAA | 0.155 |
| Alistipes putredinis | 0.152 |
| Parabacteroides merdae | 0.152 |
| Butyrivibrio crossotus | 0.152 |
| Collinsella intestinalis | 0.145 |
| Chloracidobacterium thermophilum | 0.142 |
| Massilioclostridium coli | 0.142 |
| Lachnospiraceae bacterium | 0.14 |
| 9_1_43BFAA | |
| Akkermansia sp. KLE1605 | 0.14 |
| Lachnospiraceae bacterium TF01-11 | 0.138 |
| Lachnospiraceae bacterium | 0.136 |
| 3_1_57FAA_CT1 | |
| Bacteroides sp. 9_1_42FAA | 0.136 |
| Collinsella sp. 4_8_47FAA | 0.134 |
| Ruminococcaceae bacterium D16 | 0.133 |
| Senegalimassilia anaerobia | 0.133 |
| Tyzzerella nexilis | 0.133 |
| Lachnospiraceae bacterium | 0.132 |
| 3_1_46FAA | |
| Blautia sp. SF-50 | 0.129 |
| [Clostridium] symbiosum | 0.128 |
| Holdemania sp. Marseille-P2844 | 0.124 |
| Clostridiales bacterium VE202-28 | 0.124 |
| Mycobacterium bovis | 0.123 |
| Blautia producta | 0.119 |
| Blautia sp. Marseille-P3201T | 0.117 |
| Clostridium sp. FS41 | 0.116 |
| Bacteroides massiliensis | 0.116 |
| Bacteroides sp. 4_3_47FAA | 0.116 |
| Clostridiales bacterium VE202-03 | 0.11 |
| Staphylococcus aureus | 0.109 |
| Bacteroides sp. 3_1_33FAA | 0.108 |
| Erysipelotrichaceae bacterium | 0.106 |
| 6_1_45 | |
| Anaeromassilibacillus sp. An250 | 0.104 |
| Escherichia coli | 0.102 |
| Ruthenibacterium lactatiformans | 0.099 |
| Blautia massiliensis | 0.099 |
| Lachnospiraceae bacterium | 0.099 |
| 7_1_58FAA | |
| Coprobacillus sp. 8_1_38FAA | 0.098 |
| [Clostridium] innocuum | 0.096 |
| Oscillospiraceae bacterium VE202-24 | 0.095 |
| Acetivibrio ethanolgignens | 0.095 |
| Clostridium sp. ATCC BAA-442 | 0.095 |
| Lactonifactor longoviformis | 0.094 |
| Phocea massiliensis | 0.094 |
| Oscillibacter sp. KLE 1745 | 0.093 |
| Collinsella sp. TF06-26 | 0.093 |
| Blautia schinkii | 0.091 |
| Methanosphaera stadtmanae | 0.088 |
| Clostridiales bacterium VE202-16 | 0.087 |
| Butyricicoccus pullicaecorum | 0.086 |
| Alistipes shahii | 0.086 |
| Clostridiales bacterium VE202-15 | 0.085 |
| Blautia sp. KLE 1732 | 0.084 |
| Bacteroides sp. HMSC067B03 | 0.083 |
| Clostridium sp. KLE 1755 | 0.082 |
| Bacteroides sp. 14(A) | 0.082 |
| Clostridium sp. L2-50 | 0.081 |
| [Eubacterium] dolichum | 0.08 |
| [Clostridium] citroniae | 0.08 |
| Faecalibacterium sp. An192 | 0.08 |
| Blautia hansenii | 0.078 |
| Parabacteroides goldsteinii | 0.078 |
| Faecalibacterium sp. An77 | 0.077 |
| Clostridiales bacterium VE202-27 | 0.077 |
| Blautia sp. An249 | 0.074 |
| Bifidobacterium breve | 0.072 |
| Oscillibacter sp. KLE 1728 | 0.072 |
| Lachnoclostridium sp. An138 | 0.07 |
| Phascolarctobacterium succinatutens | 0.069 |
| Lachnoclostridium sp. An14 | 0.068 |
| Clostridiales bacterium VE202-14 | 0.067 |
| Blautia sp. An81 | 0.067 |
| Pseudoflavonifractor capillosus | 0.067 |
| Pseudoflavonifractor sp. An184 | 0.066 |
| Lactobacillus rogosae | 0.065 |
| Lachnospiraceae bacterium AC2014 | 0.064 |
| Anaeromassilibacillus sp. An172 | 0.064 |
| Bacteroides caccae | 0.063 |
| Lachnoclostridium sp. An196 | 0.063 |
| Oscillibacter sp. 1-3 | 0.062 |
| Ruminococcus faecis | 0.062 |
| Lachnospiraceae bacterium | 0.062 |
| 6_1_37FAA | |
| Salmonella enterica | 0.061 |
| Drancourtella sp. An177 | 0.061 |
| Tyzzerella sp. Marseille-P3062 | 0.061 |
| Eubacterium sp. An11 | 0.059 |
| Lachnoclostridium sp. An131 | 0.059 |
| Lachnospiraceae bacterium 3-1 | 0.058 |
| Ruminococcus sp. AT10 | 0.057 |
| Eubacterium sp. 14-2 | 0.056 |
| Subdoligranulum variabile | 0.056 |
| Flavonifractor sp. An306 | 0.056 |
| Eubacterium ventriosum | 0.056 |
| Enterococcus faecalis | 0.055 |
| Marvinbryantia formatexigens | 0.055 |
| Anaeromassilibacillus sp. Marseille- | 0.054 |
| P3371 | |
| Coprobacillus sp. 8_2_54BFAA | 0.054 |
| Ruminococcus flavefaciens | 0.053 |
| Faecalibacterium sp. An58 | 0.053 |
| Faecalibacterium sp. An121 | 0.053 |
| Lachnospiraceae bacterium 3-2 | 0.052 |
| Saccharomyces cerevisiae | 0.052 |
| Flavonifractor sp. An135 | 0.052 |
| Mycobacterium tuberculosis | 0.052 |
| Dorea sp. 5-2 | 0.052 |
| Clostridia bacterium UC5.1-1D1 | 0.052 |
| Merdimonas faecis | 0.052 |
| Hespellia stercorisuis | 0.052 |
| Eubacterium plexicaudatum | 0.051 |
| Clostridium sp. DSM 4029 | 0.05 |
| Faecalibacterium sp. An122 | 0.05 |

TABLE 8
Microbial abundance in SARS-CoV-2 positive sample SG004942.

| Microbes | Relative abundances |
|---|---|
| Alistipes finegoldii | 13.187 |
| Bacteroides fragilis | 5.523 |
| [Eubacterium] eligens | 5.401 |
| Alistipes putredinis | 5.278 |
| Clostridium sp. L2-50 | 3.127 |
| [Eubacterium] siraeum | 3.084 |
| Bacteroides stercoris | 2.676 |
| Bacteroides uniformis | 2.592 |
| Bacteroides vulgatus | 2.45 |
| Faecalibacterium prausnitzii | 2.349 |
| Barnesiella intestinihominis | 2.298 |
| Lachnospiraceae bacterium | 2.17 |
| 3_1_57FAA_CT1 | |
| Parabacteroides distasonis | 1.971 |
| Blautia obeum | 1.608 |
| Gemmiger formicilis | 1.405 |
| Fusicatenibacter saccharivorans | 1.389 |
| Butyricimonas virosa | 1.094 |
| Clostridioides difficile | 0.979 |
| Blautia wexlerae | 0.973 |
| Alistipes onderdonkii | 0.937 |
| Alistipes senegalensis | 0.87 |
| Akkermansia sp. KLE1798 | 0.829 |
| Bacteroides dorei | 0.779 |
| Eisenbergiella tayi | 0.714 |
| Alistipes sp. HGB5 | 0.701 |
| [Ruminococcus] torques | 0.651 |
| Bacteroides thetaiotaomicron | 0.636 |
| Bacteroides salyersiae | 0.618 |
| Bilophila wadsworthia | 0.599 |
| Escherichia coli | 0.563 |
| Bacteroides cellulosilyticus | 0.562 |
| Parabacteroides goldsteinii | 0.544 |
| Bacteroides massiliensis | 0.535 |
| bacterium LF-3 | 0.5 |
| Bacteroides finegoldii | 0.482 |
| Parabacteroides sp. D26 | 0.465 |
| Oscillibacter sp. ER4 | 0.431 |
| Parabacteroides merdae | 0.431 |
| Alistipes shahii | 0.411 |
| Anaerostipes hadrus | 0.41 |
| Desulfovibrio sp. 6_1_46AFAA | 0.384 |
| Marvinbryantia formatexigens | 0.378 |
| Anaerotruncus colihominis | 0.361 |
| Dorea longicatena | 0.354 |
| Akkermansia sp. KLE1797 | 0.35 |
| Akkermansia sp. KLE1605 | 0.347 |
| Bacteroides sp. HMSC073E02 | 0.345 |
| Bacteroides ovatus | 0.341 |
| Desulfovibrio sp. 3_1_syn3 | 0.33 |
| Flavonifractor plautii | 0.329 |
| Coprococcus comes | 0.328 |
| Parabacteroides sp. CT06 | 0.32 |
| Lachnospiraceae bacterium 3_1_46FAA | 0.306 |
| Roseburia inulinivorans | 0.287 |
| Alistipes sp. AL-1 | 0.282 |
| [Clostridium] clostridioforme | 0.274 |
| Clostridium sp. ATCC BAA-442 | 0.274 |
| Dorea formicigenerans | 0.274 |
| Clostridiales bacterium VE202-27 | 0.269 |
| Parabacteroides sp. D25 | 0.265 |
| Clostridium sp. KLE 1755 | 0.263 |
| [Eubacterium] rectale | 0.261 |
| Intestinimonas butyriciproducens | 0.254 |
| Prevotella bivia | 0.25 |
| Hungatella hathewayi | 0.25 |
| Ruminococcaceae bacterium cv2 | 0.245 |
| Roseburia intestinalis | 0.242 |
| Ruthenibacterium lactatiformans | 0.211 |
| Bacteroides xylanisolvens | 0.21 |
| Bacteroides intestinalis | 0.204 |
| [Ruminococcus] gnavus | 0.198 |
| Collinsella aerofaciens | 0.197 |
| [Eubacterium] hallii | 0.187 |
| Alistipes indistinctus | 0.185 |
| Clostridium sp. HMSC19A11 | 0.185 |
| Blautia massiliensis | 0.185 |
| Chloracidobacterium thermophilum | 0.182 |
| Ruminococcaceae bacterium D5 | 0.174 |
| Mycobacterium bovis | 0.171 |
| [Clostridium] symbiosum | 0.168 |
| Erysipelotrichaceae bacterium 6_1_45 | 0.163 |
| Bacteroides sp. 3_1_19 | 0.16 |
| Oscillibacter sp. KLE 1745 | 0.152 |
| Adlercreutzia equolifaciens | 0.143 |
| [Clostridium] bolteae | 0.138 |
| Blautia sp. SF-50 | 0.134 |
| Butyricimonas sp. An62 | 0.131 |
| Enterococcus faecium | 0.129 |
| Bacteroides timonensis | 0.119 |
| Bacteroides sp. D20 | 0.119 |
| Blautia sp. KLE 1732 | 0.117 |
| Subdoligranulum sp. 4_3_54A2FAA | 0.116 |
| Bariatricus massiliensis | 0.115 |
| Ruminococcus sp. 5_1_39BFAA | 0.114 |
| Clostridium sp. M62/1 | 0.112 |
| Eubacterium ramulus | 0.111 |
| Parasutterella excrementihominis | 0.111 |
| Akkermansia muciniphila | 0.11 |
| Alistipes sp. CHKCI003 | 0.109 |
| Alistipes timonensis | 0.107 |
| [Clostridium] innocuum | 0.107 |
| Parabacteroides sp. 20_3 | 0.101 |
| Ruminococcaceae bacterium D16 | 0.101 |
| Blautia sp. Marseille-P3087 | 0.1 |
| Bacteroides sp. 2_1_33B | 0.1 |
| Staphylococcus aureus | 0.097 |
| Salmonella enterica | 0.096 |
| Blautia sp. Marseille-P2398 | 0.095 |
| Intestinimonas massiliensis | 0.094 |
| Roseburia faecis | 0.093 |
| Burkholderiales bacterium 1_1_47 | 0.093 |
| Alistipes sp. Marseille-P2431 | 0.092 |
| Fusicatenibacter sp. 2789STDY5834925 | 0.092 |
| Ruminococcus lactaris | 0.091 |
| Angelakisella massiliensis | 0.088 |
| Alistipes obesi | 0.086 |
| Subdoligranulum variabile | 0.086 |
| Tannerella sp. 6_1_58FAA_CT1 | 0.084 |
| Parabacteroides sp. D13 | 0.084 |
| Roseburia hominis | 0.082 |
| Alistipes sp. An31A | 0.081 |
| Coprococcus eutactus | 0.08 |
| Pseudoflavonifractor capillosus | 0.08 |
| Clostridiales bacterium VE202-28 | 0.079 |
| Bacteroides sp. 14(A) | 0.079 |
| Desulfovibrio fairfieldensis | 0.079 |
| Lachnospira pectinoschiza | 0.078 |
| Clostridium sp. HGF2 | 0.078 |
| Bilophila sp. 4_1_30 | 0.077 |
| Bacillus tequilensis | 0.076 |
| Oscillibacter sp. 1-3 | 0.076 |
| Bacteroides sp. D1 | 0.075 |
| Parabacteroides sp. AT13 | 0.074 |
| Anaeromassilibacillus sp. Marseille- | 0.074 |
| P3371 | |
| Lachnospiraceae bacterium 8_1_57FAA | 0.074 |
| Eubacterium sp. 3_1_31 | 0.073 |
| Ruminococcus sp. JC304 | 0.072 |
| Oscillibacter sp. KLE 1728 | 0.072 |
| Lachnospiraceae bacterium 5_1_63FAA | 0.071 |
| Clostridiales bacterium VE202-21 | 0.07 |
| Eubacterium coprostanoligenes | 0.07 |
| Bacteroides sp. 4_3_47FAA | 0.07 |
| Criibacterium bergeronii | 0.07 |
| Ruminococcus bromii | 0.064 |
| Alistipes sp. An66 | 0.063 |
| Coprobacter fastidiosus | 0.061 |
| Clostridium phoceensis | 0.061 |
| Bacteroides sp. 1_1_6 | 0.06 |
| Lachnoclostridium sp. An169 | 0.059 |
| [Bacteroides] pectinophilus | 0.059 |
| Lachnospiraceae bacterium 7_1_58FAA | 0.057 |
| Clostridiales bacterium VE202-01 | 0.057 |
| Oxalobacter formigenes | 0.057 |
| Bacteroides sp. 3_1_13 | 0.056 |
| Eubacterium ventriosum | 0.056 |
| Tyzzerella sp. Marseille-P3062 | 0.056 |
| Synergistes sp. 3_1_syn1 | 0.056 |
| Lactobacillus rogosae | 0.056 |
| Holdemania filiformis | 0.055 |
| Oscillibacter sp. PC13 | 0.054 |
| Ruminococcus flavefaciens | 0.054 |
| Flavonifractor sp. An10 | 0.053 |
| Odoribacter splanchnicus | 0.052 |
| Pseudoflavonifractor sp. Marseille- | 0.052 |
| P3106 | |
| Coprobacillus sp. 8_1_38FAA | 0.051 |
| Bacteroides clarus | 0.051 |
| Clostridiales bacterium | 0.051 |
| Anaerofilum sp. An201 | 0.05 |
| Dorea sp. AGR2135 | 0.05 |
| Tyzzerella nexilis | 0.05 |
| Butyrivibrio crossotus | 0.05 |

TABLE 9
Microbial abundance in SARS-CoV 2 positive sample SG005020.

| Microbes | Relative abundance |
|---|---|
| Clostridiales bacterium VE202-16 | 0.05 |
| Faecalibacterium sp. An192 | 0.05 |
| Bacteroides sp. 3_1_23 | 0.051 |
| Anaerofilum sp. An201 | 0.052 |
| Ruminococcus flavefaciens | 0.055 |
| Clostridium sp. AT4 | 0.055 |
| Parabacteroides sp. D13 | 0.055 |
| Ruminococcaceae bacterium cv2 | 0.055 |
| Collinsella sp. 4_8_47FAA | 0.056 |
| Bacteroides cellulosilyticus | 0.056 |
| Enterococcus faecalis | 0.057 |
| Collinsella sp. TF06-26 | 0.057 |
| [Clostridium] lactatifermentans | 0.057 |
| Erysipelotrichaceae bacterium 2_2_44A | 0.057 |
| Acetivibrio ethanolgignens | 0.058 |
| Pseudoflavonifractor sp. An184 | 0.06 |
| Clostridiales bacterium | 0.061 |
| Angelakisella massiliensis | 0.062 |
| Eubacterium ventriosum | 0.063 |
| Pseudoflavonifractor sp. Marseille- | 0.064 |
| P3106 | |
| [Eubacterium] siraeum | 0.065 |
| Lachnospiraceae bacterium 3_1_46FAA | 0.067 |
| Desulfovibrio fairfieldensis | 0.069 |
| Blautia sp. Marseille-P2398 | 0.069 |
| Ruminococcus sp. 5_1_39BFAA | 0.071 |
| Tyzzerella nexilis | 0.073 |
| Oscillibacter sp. PC13 | 0.074 |
| Parabacteroides sp. CT06 | 0.079 |
| [Clostridium] bolteae | 0.079 |
| Prevotella sp. P4-119 | 0.08 |
| Intestinimonas massiliensis | 0.081 |
| Prevotellamassilia timonensis | 0.081 |
| Prevotella sp. 885 | 0.081 |
| Lactobacillus rogosae | 0.084 |
| Butyricicoccus pullicaecorum | 0.084 |
| Bacteroides sp. 9_1_42FAA | 0.084 |
| Klebsiella pneumoniae | 0.086 |
| Oscillibacter sp. KLE 1728 | 0.093 |
| Ruminococcaceae bacterium D16 | 0.094 |
| Bacteroides sp. HMSC068A09 | 0.096 |
| Oscillospiraceae bacterium VE202-24 | 0.097 |
| Mycobacterium bovis | 0.099 |
| Pseudoflavonifractor capillosus | 0.101 |
| Bacteroides clarus | 0.102 |
| Butyricimonas virosa | 0.102 |
| Salmonella enterica | 0.103 |
| Oscillibacter sp. 1-3 | 0.104 |
| Alistipes onderdonkii | 0.105 |
| Bacteroides sp. 4_3_47FAA | 0.106 |
| [Clostridium] innocuum | 0.107 |
| Clostridiales bacterium VE202-03 | 0.107 |
| Hungatella hathewayi | 0.107 |
| Bacteroides sp. 4_1_36 | 0.109 |
| Subdoligranulum variabile | 0.109 |
| Blautia sp. KLE 1732 | 0.11 |
| Subdoligranulum sp. 4_3_54A2FAA | 0.111 |
| Eubacterium ramulus | 0.113 |
| Ruthenibacterium lactatiformans | 0.113 |
| Paraprevotella xylaniphila | 0.115 |
| Parabacteroides sp. 20_3 | 0.115 |
| Clostridium butyricum | 0.116 |
| Lachnospiraceae bacterium 7_1_58FAA | 0.117 |
| Enterococcus faecium | 0.119 |
| Sutterella sp. KLE1602 | 0.12 |
| Bacteroides sp. D20 | 0.121 |
| Bacteroides xylanisolvens | 0.121 |
| Butyrivibrio crossotus | 0.121 |
| Blautia massiliensis | 0.127 |
| Bacteroides finegoldii | 0.129 |
| bacterium LF-3 | 0.132 |
| [Clostridium] symbiosum | 0.134 |
| Oscillibacter sp. KLE 1745 | 0.135 |
| Coprococcus eutactus | 0.136 |
| Staphylococcus aureus | 0.143 |
| Bacteroides sp. 3_1_33FAA | 0.144 |
| Escherichia coli | 0.148 |
| [Ruminococcus] gnavus | 0.148 |
| Blautia sp. SF-50 | 0.152 |
| Bacteroides sp. 3_1_19 | 0.152 |
| Alistipes finegoldii | 0.154 |
| Lachnospiraceae bacterium TF01-11 | 0.156 |
| Akkermansia sp. KLE1798 | 0.159 |
| Clostridium sp. M62/1 | 0.167 |
| Alistipes senegalensis | 0.171 |
| Oxalobacter formigenes | 0.173 |
| Clostridium sp. ATCC BAA-442 | 0.185 |
| Roseburia faecis | 0.198 |
| Lachnospira pectinoschiza | 0.204 |
| Dorea formicigenerans | 0.206 |
| Bacteroides salyersiae | 0.215 |
| Bilophila wadsworthia | 0.218 |
| Bacteroides thetaiotaomicron | 0.218 |
| Blautia sp. Marseille-P3087 | 0.233 |
| Coprococcus comes | 0.234 |
| Desulfovibrio sp. 3_1_syn3 | 0.239 |
| [Clostridium] clostridioforme | 0.246 |
| Clostridiales bacterium KLE1615 | 0.246 |
| Anaerotruncus colihominis | 0.253 |
| [Ruminococcus] torques | 0.259 |
| Intestinimonas butyriciproducens | 0.261 |
| Methanobrevibacter smithii | 0.271 |
| Desulfovibrio sp. 6_1_46AFAA | 0.275 |
| Bacteroides stercoris | 0.282 |
| Prevotella sp. KHD1 | 0.285 |
| Collinsella aerofaciens | 0.293 |
| Butyricimonas sp. An62 | 0.293 |
| Roseburia hominis | 0.307 |
| Flavonifractor plautii | 0.317 |
| Prevotella sp. P4-98 | 0.326 |
| Bacteroides faecis | 0.342 |
| [Eubacterium] hallii | 0.354 |
| Clostridium phoceensis | 0.362 |
| Odoribacter splanchnicus | 0.367 |
| Ruminococcus lactaris | 0.382 |
| Bacteroides sp. 3_1_40A | 0.386 |
| Bacteroides caccae | 0.394 |
| Blautia obeum | 0.404 |
| Fusicatenibacter saccharivorans | 0.423 |
| Alistipes indistinctus | 0.444 |
| Dorea longicatena | 0.484 |
| Bacteroides intestinalis | 0.505 |
| Blastocystis hominis | 0.508 |
| Roseburia inulinivorans | 0.514 |
| Anaerostipes hadrus | 0.517 |
| Gemmiger formicilis | 0.518 |
| Odoribacter laneus | 0.533 |
| Roseburia intestinalis | 0.534 |
| Blautia wexlerae | 0.555 |
| Eisenbergiella tayi | 0.664 |
| [Bacteroides] pectinophilus | 0.728 |
| Sutterella wadsworthensis | 0.793 |
| Paraprevotella clara | 0.827 |
| Alistipes obesi | 0.852 |
| Akkermansia muciniphila | 0.857 |
| Clostridioides difficile | 0.94 |
| Ruminococcus bicirculans | 0.962 |
| Bacteroides dorei | 1.077 |
| Alistipes shahii | 1.109 |
| Oscillibacter sp. ER4 | 1.186 |
| Catenibacterium mitsuokai | 1.19 |
| [Eubacterium] rectale | 1.2 |
| Alistipes putredinis | 1.212 |
| [Eubacterium] eligens | 1.27 |
| Bacteroides massiliensis | 1.35 |
| Bacteroides fragilis | 1.367 |
| Parabacteroides merdae | 1.416 |
| Lachnospiraceae bacterium | 1.478 |
| 3_1_57FAA_CT1 | |
| Bacteroides uniformis | 1.509 |
| Barnesiella intestinihominis | 1.828 |
| Parabacteroides distasonis | 2.592 |
| Dialister succinatiphilus | 2.658 |
| Bacteroides vulgatus | 2.702 |
| Bacteroides ovatus | 3.506 |
| Clostridium sp. L2-50 | 3.673 |
| Faecalibacterium prausnitzii | 11.757 |
| Prevotella copri | 16.227 |

### Example 2

### Detection of Opportunistic Microbes in the Gut

Using the method of the invention, a subject infected with SARS-CoV-2 was detected with the assay set forth in Example 1. The subject was identified as a COVID-19 “long hauler” and metagenomic analysis performed by the method of the invention identified the subject as having a high abundance level of Serratia marcescen in their gut.

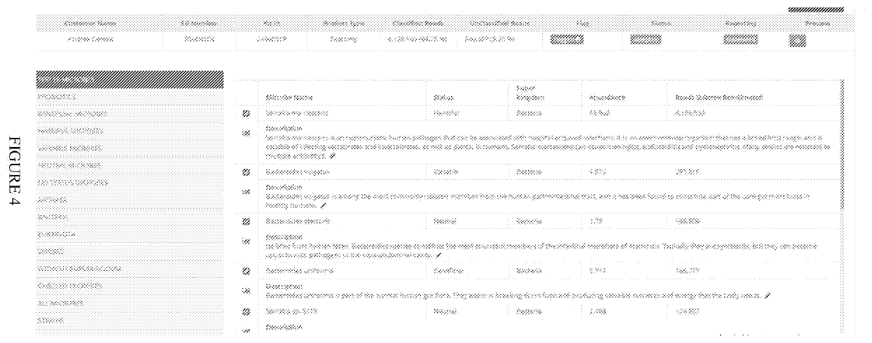

Serratia marcescen, is an opportunist pathogen (harmful microbe) that can be associated with hospital-acquired infections (FIG. 4). The microbe was detected at an abundance level above 66% in the subject's gut when a sample of the subject's stool was subjected to WGS and subsequent metagenomics analysis. This pathogen has been associated with hospital acquired pneumonia through medical devices like ventilator and belongs to the family Enterobacteriaceae, which is known to cause urinary and respiratory tract infections and exhibits antibiotics resistance.

By treating the subject with a therapeutic composition of the present invention, it is expected that levels of this microbe can be reduced to treat and/or otherwise ameliorate infection and associated disorders caused by infection of the microbe.

Although the invention has been described, it will be understood that modifications and variations are encompassed within the spirit and scope of the invention. Accordingly, the invention is limited only by the following claims.

What is claimed is:

1. A method comprising:
detecting exposure to a pathogen in a subject;
analyzing the microbiome of the subject and identifying opportunistic pathogens in the subject that indicate a dysbiosis or potential onset/recovery of disease symptoms; and
optionally treating the subject with a therapeutic composition.

2. The method of claim 1, wherein the pathogen is a bacterial, fungal, parasitic or viral pathogen.

3. The method of claim 2, wherein the pathogen is a viral pathogen.

4. The method of claim 3, wherein the viral pathogen is a coronavirus, Zika virus, influenza virus or Ebola virus.

5. The method of claim 4, wherein the coronavirus is selected from Coronavirus Disease 2019 (COVID-19), SARS associated coronavirus (SARS-CoV), or Middle East respiratory syndrome coronavirus (MERS-CoV).

6. The method of claim 5, wherein the coronavirus is SARS-CoV-2.

7. The method of claim 1, wherein the disease symptoms are respiratory complications and/or dysbiosis.

8. The method of claim 1, wherein the therapeutic composition comprises a probiotic, pre-biotic and/or metabolite of the gut microbiome.

9. The method of claim 8, wherein the therapeutic composition is customized and based on classification of the identified opportunistic pathogens.

10. The method of claim 8, wherein the probiotic comprises one or more of Bacillus coagulans, Bacillus indicus, Bacillus lichenformis, Bacillus subtilis, Bifidobacterium animalis, Bifidobacterium bifidum, Bifidobacterium breve, Bifidobacterium coagilans, Bifidobacterium infantis, Bifidobacterium lactis, Bifidobacterium longum, Bifidobacterium subtilis, Enterococcus faecium, Lactobacillus acidophilus, Lactobacillus bulgaricus, Lactobacillus casei, Lactobacillus delbrueckii, Lactobacillus gasseri, Lactobacillus helveticus, Lactobacillus lactis, Lactobacillus paracasei, Lactobacillus plantarum, Lactobacillus reuteri, Lactobacillus rhamnosus, Lactobacillus salivarius, Saccharomyces boulardii, Streptococcus thermophiles, Lactobacillus buchneri, Lactobacillus fermentum, Lactobacillus crispatus, Bifidobacterium catenulatum, and Bifidobacterium pseudocatenulatum.

11. The method of claim 8, wherein the probiotic comprises one or more organisms set forth in Tables 1 and 3.

12. The method of claim 1, wherein the therapeutic composition comprises an antibiotic, an antiviral agent, plasma, hormone, steroid, corticosteroid, small organic compound, or any combination thereof.

13. The method of claim 12, wherein the therapeutic composition further comprises a probiotic, pre-biotic and/or a metabolite of the gut microbiome.

14. The method of claim 13, wherein the probiotic or pre-biotic is a natural product or extract thereof.

15. The method of claim 1, wherein the opportunistic pathogen is from the gut of the subject.

16. The method of claim 1, wherein the pathogen is detected using a PCR based method.

17. The method of claim 1, wherein the pathogen is detected via a nucleic acid selected from DNA or RNA.

18. The method of claim 17, wherein the pathogen is detected via RNA using RT-PCR.

19. The method of claim 1, further comprising detecting the pathogen using an IgG/IgM specific antibody test.

20. The method of claim 1, wherein the therapeutic composition targets one or more of a spike surface protein, a cell or virus membrane protein or receptor such as ACE2 and endocytosis, an intra or extracellular signaling pathway such as ACE2 MAP2K, proteolysis such as 3C-like protease inhibition, translation of RNA from virus and RNA replication, and packaging of virus and release from cells.

21. The method of claim 1, wherein the opportunistic pathogen is selected from one listed in FIGS. 1-4 or Tables 6-9.

22. A therapeutic composition comprising:
a) a natural product or derivative thereof; and optionally
b) a probiotic comprising a microorganism.

23. The therapeutic composition of claim 22, wherein the natural product or derivative thereof is selected from the group consisting of bioflavonoids, metabolites, antioxidants, vitamins and minerals.

24. The therapeutic composition of claim 22, wherein the microorganism is selected from one or more organisms set forth in Tables 1 and 3.

25. The therapeutic composition of claim 22, wherein the natural product or derivative thereof is a plant or plant extract.

26. The therapeutic composition of claim 25, wherein the natural product or derivative thereof is derived from a fruit, berry, vegetable, tea, grass, root, seed, leaf and/or flower.

27. The therapeutic composition of claim 25, wherein the natural product or derivative thereof is derived from a citrus plant or fruit thereof.

28. The therapeutic composition of claim 27, wherein the natural product or derivative thereof is Vitamin C and/or ascorbic acid.

29. The therapeutic composition of claim 27, wherein the natural product or derivative thereof comprises hesperidin or analog thereof.

30. The therapeutic composition of claim 25, wherein the natural product or derivative thereof comprises quercetin or an analog thereof.

31. The therapeutic composition of claim 25, wherein the natural product or derivative thereof is derived from a tea plant.

32. The therapeutic composition of claim 31, wherein the tea is green tea, black tea or puer tea.

33. The therapeutic composition of claim 32, wherein the green tea is matcha.

34. The therapeutic composition of any of claims 31-33, wherein the natural product or derivative thereof comprises Epigallocatechin Gallate (EGCG).

35. The therapeutic composition of any of claims 31-32, wherein the natural product or derivative thereof comprises theaflavin-3,3′-digallate (TF3).

36. The therapeutic composition of claim 22, wherein the natural product or derivative thereof is an anti-inflammatory and/or a hyaluronic acid blocker.

37. The therapeutic composition of claim 22, wherein the microorganism is selected from one or more organisms set forth in Tables 1 and 3, and the natural product or derivative thereof comprises hesperidin, Vitamin C, ascorbic acid or other citrus extract, quercetin or an analog thereof, EGCG, TF3 or any combination thereof.

38. The therapeutic composition of claim 37, wherein the ECGC is present in the form of green tea powder or as a green tea extract.

39. The therapeutic composition of claim 38, wherein the green tea is matcha.

40. The therapeutic composition of claim 37, wherein the TF3 is present in the form of black tea powder or as a black tea extract.

41. The therapeutic composition of any of claims 22 to 40, further comprising a therapeutic agent selected from the group consisting of an anti-inflammatory and/or hyaluronic acid blocker, antibiotic, an antiviral agent, plasma, hormone, steroid, corticosteroid, small organic compound, and any combination thereof.

42. A method comprising administering to a subject the therapeutic composition of any of claims 22 to 41.

43. The method of claim 42, wherein the subject is infected, or has previously been infected with a pathogen.

44. The method of claim 43, wherein the pathogen is a bacterial, fungal, parasitic or viral pathogen.

45. The method of claim 44, wherein the pathogen is a viral pathogen.

46. The method of claim 45, wherein the viral pathogen is a coronavirus, Zika virus, influenza virus or Ebola virus.

47. The method of claim 46, wherein the coronavirus is selected from Coronavirus Disease 2019 (COVID-19), SARS associated coronavirus (SARS-CoV), or Middle East respiratory syndrome coronavirus (MERS-CoV).

48. The method claim 47, wherein the coronavirus is SARS-CoV-2.

49. The method of any of claims 1 to 21, further comprising administering to the subject the therapeutic composition of any of claims 22 to 41.

50. The method of claim 49, wherein the subject is administered the therapeutic composition after being exposed to, and/or diagnosed as being infected with the pathogen.

51. The method of claim 50, wherein the subject is administered the therapeutic composition daily for about 3 to 21 days.

52. The method of claim 50, wherein the subject is retested for infection after about 3 to 12 days.

53. A method comprising:
screening a subject for a previous exposure to a virus using an IgG/IgM specific antibody assay,
wherein if the subject is IgM negative, the subject is screened for the virus via a PCR based assay and administered the therapeutic composition of any of claims 22 to 41 where the PCR based assay is positive and then rescreened using the IgG/IgM specific antibody assay after about 3 to 21 days, and
wherein if the subject is IgM positive, the subject is administered the therapeutic composition of any of claims 22 to 41 and then rescreened using the IgG/IgM specific antibody assay after about 3 to 21 days.

54. A method comprising:
screening a subject for a viral infection using a PCR based assay,
wherein if the PCR based assay is positive the subject is administered the therapeutic composition of any of claims 22 to 35 and then rescreened using the PCR based assay after about 3 to 21 days, and
wherein if the PCR based assay is negative, the subject is screened for a previous exposure to the virus using an IgG/IgM specific antibody assay, wherein if the subject is IgM negative, the subject is screened for risk of infecting another subject via a PCR based test and administered the therapeutic composition of any of claims 22 to 41 where the PCR based assay is positive and then rescreened using the IgG/IgM specific antibody assay after about 3 to 12 days, and wherein if the subject is IgM positive, the subject is administered the therapeutic composition of any of claims 22 to 41 and the rescreened using the IgG/IgM specific antibody assay after about 3 to 21 days.

55. The method of any of claims 53 or 54, further comprising treating the subject with a treatment as listed in Table 4.

56. The method of any of claims 53 or 54, wherein the virus is SARS-CoV-2.

57. A method of detecting SARS-CoV-2 in a biological sample, the method comprising:
a) obtaining a biological sample comprising ribonucleic acids;
b) reverse transcribing the ribonucleic acids to obtain cDNA;
c) contacting the cDNA with a first and/or second primer set, and a DNA polymerase to produce a first and/or second PCR product, wherein the first primer set comprises SEQ ID NOs: 1 and 2 and the second primer set comprises SEQ ID NOs: 5 and 6;
d) hybridizing to the first PCR product a first nucleic acid probe comprising SEQ ID NO: 3 and/or SEQ ID NO: 4, and/or hybridizing to the second PCR product a second nucleic acid probe comprising SEQ IN NO: 7 and/or 8; and
e) detecting hybridization of the first nucleic acid probe to the first PCR product and/or detecting hybridization of the second nucleic acid probe to the second PCR product,
wherein hybridization of the first nucleic acid probe to the first PCR product, hybridization of the second nucleic acid probe to the second PCR product, is indicative of the presence of SARS-CoV-2 nucleic acids in the biological sample.

58. The method of claim 57, further comprising:
contacting the cDNA with a control primer set, and a DNA polymerase to produce a control PCR product, wherein the control primer set comprises SEQ ID NOs: 9 and 10;
hybridizing to the control PCR product a control nucleic acid probe comprising SEQ ID NO: 11 and/or SEQ ID NO: 12; and
detecting hybridization of the control nucleic acid probe to the control PCR product.

59. The method of claim 57, wherein the biological sample is blood, plasma, sweat, nasal discharge, phlegm, saliva, sweat, tears, urine, feces, gut material, cerebrospinal fluid or vomit.

60. The method of claim 59, wherein the biological sample is feces.

61. A kit comprising:
a) first and/or second primer set, wherein the first primer set comprises SEQ ID NOs: 1 and 2 and the second primer set comprises SEQ ID NOs: 5 and 6;
b) a first nucleic acid probe comprising SEQ ID NO: 3 and/or SEQ ID NO: 4, and/or a second nucleic acid probe comprising SEQ IN NO: 7 and/or 8; and optionally
c) reagents for conducting a reverse transcription-polymerase chain reaction using a) and b).

62. The kit of claim 61, further comprising a control primer set, wherein the control primer set comprises SEQ ID NOs: 9 and 10, and a control nucleic acid probe comprising SEQ ID NO: 11 and/or SEQ ID NO: 12.


SEQUENCE LISTING (12 sequences)

SEQ ID NO: 1 - DNA, 20 bp, Artificial Sequence
  Description of Artificial Sequence Synthetic primer
gaccccaaaatcagcgaaat

SEQ ID NO: 2 - DNA, 24 bp, Artificial Sequence
  Description of Artificial Sequence Synthetic primer
tctggttactgccagttgaatctg

SEQ ID NO: 3 - DNA, 24 bp, Artificial Sequence
  Description of Artificial Sequence Synthetic probe
accccgcattacgtttggtggacc

SEQ ID NO: 4 - DNA, 15 bp, Artificial Sequence
  Description of Artificial Sequence Synthetic probe
tacgtttggtggacc

SEQ ID NO: 5 - DNA, 20 bp, Artificial Sequence
  Description of Artificial Sequence Synthetic primer
ttacaaacattggccgcaaa

SEQ ID NO: 6 - DNA, 18 bp, Artificial Sequence
  Description of Artificial Sequence Synthetic primer
gcgcgacattccgaagaa

SEQ ID NO: 7 - DNA, 23 bp, Artificial Sequence
  Description of Artificial Sequence Synthetic probe
acaatttgcccccagcgcttcag

SEQ ID NO: 8 - DNA, 14 bp, Artificial Sequence
  Description of Artificial Sequence Synthetic probe
ccccagcgcttcag

SEQ ID NO: 9 - DNA, 19 bp, Artificial Sequence
  Description of Artificial Sequence Synthetic primer
agatttggacctgcgagcg

SEQ ID NO: 10 - DNA, 20 bp, Artificial Sequence
  Description of Artificial Sequence Synthetic primer
gagcggctgtctccacaagt

SEQ ID NO: 11 - DNA, 23 bp, Artificial Sequence
  Description of Artificial Sequence Synthetic probe
ttctgacctgaaggctctgcgcg

SEQ ID NO: 12 - DNA, 14 bp, Artificial Sequence
  Description of Artificial Sequence Synthetic probe
gaaggctctgcgcg